(19) United States
(12) Patent Application Publication
(10) Pub. No.: US 2022/0221397 A1
(43) Pub. Date: Jul. 14, 2022

(54) GAS DETECTION SYSTEM

(71) Applicant: KYOCERA Corporation, Kyoto-shi, Kyoto (JP)

(72) Inventors: Shinichi ABE, Uji-shi, Kyoto (JP); Ryo UENO, Kyotanabe-shi, Kyoto (JP)

(21) Appl. No.: 17/613,301

(22) PCT Filed: May 28, 2020
(86) PCT No.: PCT/JP2020/021161

§ 371 Date: Nov. 22, 2021

(30) Foreign Application Priority Data
May 29, 2019 (JP) 2019-100598
May 29, 2019 (JP) 2019-100644

(51) Int. Cl. G01N 21/31; G01N 33/00

(52) U.S. Cl. CPC G01N 21/3103; G01N 33/0027; G01N 33/0073

(57) ABSTRACT

A gas detection system includes a sensor unit that outputs a signal corresponding to a concentration of a specific gas, a concentration unit having therein an adsorbent that adsorbs a gas to be detected, a supply unit capable of supplying a sample gas and a purge gas to the concentration unit, a heater capable of heating the adsorbent, and a control unit. The control unit controls the supply unit so that the sample gas passes through the concentration unit, and then controls the supply unit so that the purge gas passes through the concentration unit while controlling the heater so that a temperature of the adsorbent increases.

### CROSS REFERENCE TO RELATED APPLICATIONS

The present application claims priority to Japanese Patent Application No. 2019-100598 filed in Japan on May 29, 2019 and Japanese Patent Application No. 2019-100644 filed in Japan on May 29, 2019, the entire disclosure of which is incorporated herein by reference.

### TECHNICAL FIELD

The present disclosure relates to a gas detection system.

### BACKGROUND ART

In the related art, there is known a system for detecting an odoriferous gas generated from feces discharged by a subject (for example, PTL 1).

### CITATION LIST

### Patent Literature

PTL 1: Japanese Unexamined Patent Application Publication No. 2016-142584

### SUMMARY OF INVENTION

A gas detection system according to an embodiment of the present disclosure includes:

a sensor unit that outputs a signal corresponding to a concentration of a specific gas;

a concentration unit having therein an adsorbent that adsorbs a gas to be detected;

a supply unit capable of supplying a sample gas and a purge gas to the concentration unit;

a heater capable of heating the adsorbent; and

a control unit that controls the supply unit so that the sample gas passes through the concentration unit and then controls the supply unit so that the purge gas passes through the concentration unit while controlling the heater so that a temperature of the adsorbent increases, wherein

the control unit

stops passage of the purge gas to the concentration unit from a first point in time to a second point in time later than the first point in time, the first point in time being a point in time before or at which the temperature of the adsorbent reaches a desorption temperature of the gas to be detected, and, after the second point in time, performs control so that the purge gas passes through the concentration unit and is supplied to the sensor unit together with the gas to be detected in the concentration unit.

### BRIEF DESCRIPTION OF DRAWINGS

FIG. 1 is an external view of a gas detection system according to a first embodiment of the present disclosure.

FIG. 2 is a schematic diagram of the inside of a housing of the gas detection system illustrated in FIG. 1.

FIG. 3 is a functional block diagram of the gas detection system illustrated in FIG. 1.

FIG. 4 is a schematic graph of the concentration of a gas desorbed from an adsorbent adsorbing a predetermined gas, which is detected with a change in the temperature of the adsorbent.

FIG. 5 is a timing chart of an example operation of the gas detection system illustrated in FIG. 1.

FIG. 6 is a flowchart of an example operation of the gas detection system illustrated in FIG. 1 during gas concentration.

FIG. 7 is a flowchart of an example operation of the gas detection system illustrated in FIG. 1 during detection of the type and concentration of a gas.

FIG. 8 is timing chart of an example operation of a gas detection system according to a second embodiment of the present disclosure.

FIG. 9 is a timing chart describing another example of a first point in time and a second point in time in the present disclosure.

FIG. 10 is a functional block diagram of a gas detection system according to a modification of the first embodiment and the second embodiment of the present disclosure.

FIG. 11 is an external view of a gas detection system according to a third embodiment of the present disclosure.

FIG. 12 is a schematic diagram of the inside of a housing of the gas detection system illustrated in FIG. 11.

FIG. 13 is a functional block diagram of the gas detection system illustrated in FIG. 11.

FIG. 14 is a schematic graph of the concentration of a gas desorbed from an adsorbent adsorbing a predetermined gas, which is detected with a change in the temperature of the adsorbent.

FIG. 15 is a sectional view of the adsorbent in a concentration tank illustrated in FIG. 12.

FIG. 16 is a timing chart of an example operation of the gas detection system illustrated in FIG. 11.

FIG. 17 is a flowchart of an example operation of the gas detection system illustrated in FIG. 11 during gas concentration.

FIG. 18 is a flowchart of an example operation of the gas detection system illustrated in FIG. 11 during detection of the type and concentration of a gas.

FIG. 19 is timing chart of an example operation of a gas detection system according to a fourth embodiment of the present disclosure.

FIG. 20 is a flowchart of an example operation of the gas detection system according to the fourth embodiment of the present disclosure during gas concentration.

FIG. 21 is a schematic graph illustrating the relationship between the temperature of an adsorbent and the concentration of a gas desorbed from the adsorbent in a fifth embodiment of the present disclosure.

FIG. 22 is a functional block diagram of a gas detection system according to a modification of the third embodiment to the fifth embodiment of the present disclosure.

### DESCRIPTION OF EMBODIMENTS

A conventional system needs to improve the gas detection performance and the like.

The present disclosure relates to providing a gas detection system with improved gas detection performance and the like.

According to an embodiment of the present disclosure, a gas detection system with improved gas detection performance and the like can be provided.

Embodiments according to the present disclosure will be described hereinafter with reference to the drawings. The drawings are schematic illustrations.

### First Embodiment

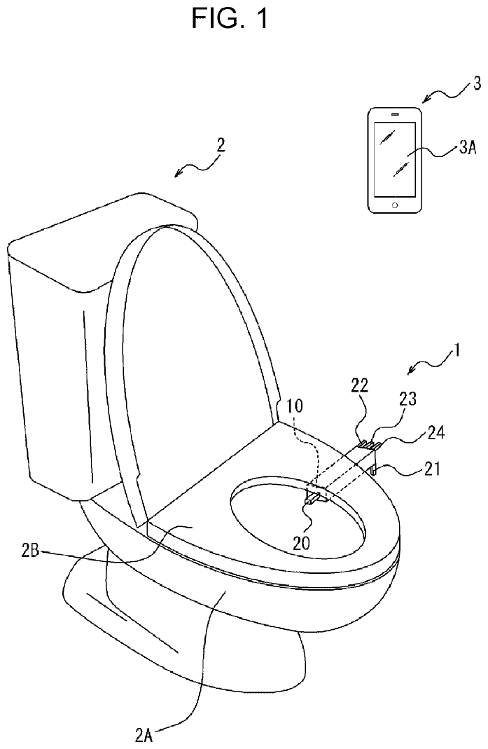

As illustrated in FIG. 1, a gas detection system 1 is installed in a toilet 2. The toilet 2 may be, but is not limited to, a flush toilet. The toilet 2 includes a toilet bowl 2A and a toilet seat 2B. The gas detection system 1 may be installed in any portion of the toilet 2. In one example, as illustrated in FIG. 1, the gas detection system 1 may be arranged from between the toilet bowl 2A and the toilet seat 2B to the outside of the toilet 2. A portion of the gas detection system 1 may be embedded inside the toilet seat 2B. The subject can discharge feces into the toilet bowl 2A. The gas detection system 1 can acquire a gas generated from the feces discharged into the toilet bowl 2A as a sample gas. The gas detection system 1 can detect the type of a gas contained in the sample gas, the concentration of the gas, and so on. The gas detection system 1 can transmit the detection results and so on to an electronic device 3. The gas detection system 1 as illustrated in FIG. 1 is also referred to as a “gas detection device”.

The uses of the gas detection system 1 are not limited to the use described above. For example, the gas detection system 1 may be installed in a refrigerator. In this case, the gas detection system 1 can acquire a gas generated from food as a sample gas. In another use, for example, the gas detection system 1 may be installed in a factory or a laboratory. In this case, the gas detection system 1 can acquire a gas generated from a chemical or the like as a sample gas.

The toilet 2 can be installed in a toilet room in a house, a hospital, or the like. The toilet 2 can be used by the subject. As described above, the toilet 2 includes the toilet bowl 2A and the toilet seat 2B. The subject can discharge feces into the toilet bowl 2A.

The electronic device 3 is, for example, a smartphone used by the subject. However, the electronic device 3 is not limited to a smartphone. The electronic device 3 may be any electronic device. When brought into the toilet room by the subject, as illustrated in FIG. 1, the electronic device 3 can be present in the toilet room. However, for example, when the subject does not bring the electronic device 3 into the toilet room, the electronic device 3 may be present outside the toilet room. The electronic device 3 can receive the detection results from the gas detection system 1 via wireless communication or wired communication. The electronic device 3 can display the received detection results on a display unit 3A. The display unit 3A may include a display capable of displaying characters and the like, and a touch screen capable of detecting contact of a finger of the user (subject) or the like. The display may include a display device such as a liquid crystal display (LCD), an organic EL display (OELD: Organic Electro-Luminescence Display), or an inorganic EL display (IELD: Inorganic Electro-Luminescence Display). The detection method of the touch screen may be any method such as a capacitance method, a resistance film method, a surface acoustic wave method, an ultrasonic method, an infrared method, an electromagnetic induction method, or a load detection method.

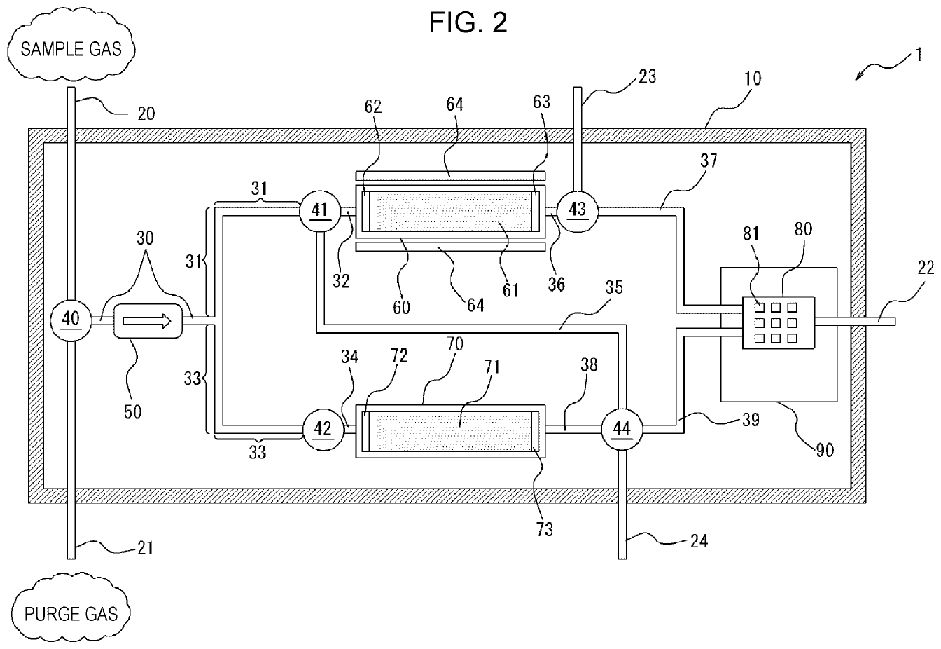

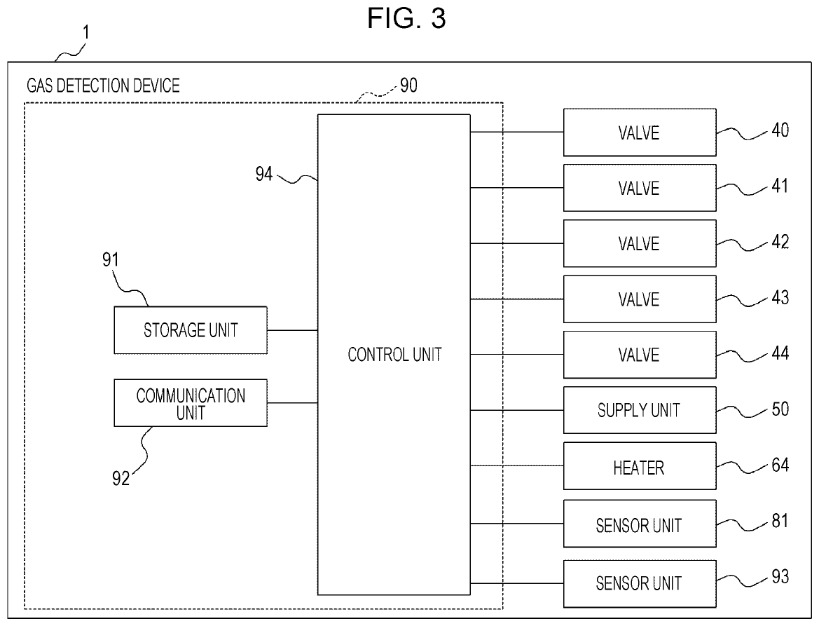

As illustrated in FIG. 2, the gas detection system 1 includes a housing 10, inflow paths 20 and 21, and discharge paths 22, 23, and 24. The discharge path 22, the discharge path 23, and the discharge path 24 may merge in any location. The gas detection system 1 includes flow paths 30, 31, 32, 33, 34, 35, 36, 37, 38, and 39, valves 40, 41, 42, 43, and 44, and a supply unit 50. The gas detection system 1 includes a concentration tank 60 serving as a concentration unit, a storage tank 70 serving as a reservoir, a chamber 80, and a circuit board 90 serving as a circuit unit. As illustrated in FIG. 3, the gas detection system 1 includes, in the circuit board 90, a storage unit 91, a communication unit 92, and a control unit 94. The gas detection system 1 includes a sensor unit 93.

The housing 10 houses various components of the gas detection system 1. The housing 10 may be made of any material. For example, the housing 10 may be made of a material such as metal or resin.

As illustrated in FIG. 1, the inflow path 20 can be exposed to the inside of the toilet bowl 2A. A portion of the inflow path 20 may be embedded in the toilet seat 2B. A gas generated from feces discharged into the toilet bowl 2A flows into the inflow path 20 as a sample gas. The sample gas flowing into the inflow path 20 is supplied to the concentration tank 60 through the flow paths 30, 31, and 32. As illustrated in FIG. 1, one end of the inflow path 20 may be directed to the inside of the toilet bowl 2A. As illustrated in FIG. 2, the other end of the inflow path 20 may be connected to the valve 40. The inflow path 20 may be constituted by a tubular member such as a resin tube or a metal or glass pipe.

As illustrated in FIG. 1, the inflow path 21 can be exposed to the outside of the toilet bowl 2A. A portion of the inflow path 21 may be embedded in the toilet seat 2B. For example, air in the toilet room, which is outside the toilet bowl 2A, flows into the inflow path 21 as a purge gas. The purge gas flowing into the inflow path 21 is supplied to the storage tank 70 through the flow paths 30, 33, and 34. As illustrated in FIG. 1, one end of the inflow path 21 may be directed to the outside of the toilet 2. As illustrated in FIG. 2, the other end of the inflow path 21 may be connected to the valve 40. The inflow path 21 may be constituted by a tubular member such as a resin tube or a metal or glass pipe.

As illustrated in FIG. 1, a portion of the discharge path 22 can be exposed to the outside of the toilet bowl 2A. The discharge path 22 as illustrated in FIG. 2 discharges the exhaust from the chamber 80 to the outside. This exhaust can contain the sample gas and the purge gas, which have been subjected to detection processing. As illustrated in FIG. 1, one end of the discharge path 22 may be directed to the outside of the toilet 2. As illustrated in FIG. 2, the other end of the discharge path 22 may be connected to the chamber 80. The discharge path 22 may be constituted by a tubular member such as a resin tube or a metal or glass pipe.

As illustrated in FIG. 1, a portion of the discharge path 23 can be exposed to the outside of the toilet bowl 2A. The discharge path 23 as illustrated in FIG. 2 discharges the exhaust from the concentration tank 60 to the outside. This exhaust includes a gas not to be detected, which is generated in a concentration process of the sample gas described below. As illustrated in FIG. 1, one end of the discharge path 23 may be directed to the outside of the toilet 2. As illustrated in FIG. 2, the other end of the discharge path 23 may be connected to the valve 43. The discharge path 23 may be constituted by a tubular member such as a resin tube or a metal or glass pipe.

As illustrated in FIG. 1, a portion of the discharge path 24 can be exposed to the outside of the toilet bowl 2A. The discharge path 24 as illustrated in FIG. 2 discharges the residual gas or the like from the storage tank 70 to the outside. As illustrated in FIG. 1, one end of the discharge path 24 may be directed to the outside of the toilet 2. As illustrated in FIG. 2, the other end of the discharge path 24 may be connected to the valve 44. The discharge path 24 may be constituted by a tubular member such as a resin tube or a metal or glass pipe.

As illustrated in FIG. 2, one end of the flow path 30 is connected to the valve 40. The other end of the flow path 30 is connected to one end of the flow path 31 and one end of the flow path 33. The one end of the flow path 31 is connected to the other end of the flow path 30. The other end of the flow path 31 is connected to the valve 41. One end of the flow path 32 is connected to the valve 41. The other end of the flow path 32 is connected to an inlet portion of the concentration tank 60. The one end of the flow path 33 is connected to the other end of the flow path 30. The other end of the flow path 33 is connected to the valve 42. One end of the flow path 34 is connected to the valve 42. The other end of the flow path 34 is connected to an inlet portion of the storage tank 70. One end of the flow path 35 is connected to the valve 41. The other end of the flow path 35 is connected to the valve 44. One end of the flow path 36 is connected to an outlet portion of the concentration tank 60. The other end of the flow path 36 is connected to the valve 43. One end of the flow path 37 is connected to the valve 43. The other end of the flow path 37 is connected to the chamber 80. One end of the flow path 38 is connected to an outlet portion of the storage tank 70. The other end of the flow path 38 is connected to the valve 44. One end of the flow path 39 is connected to the valve 44. The other end of the flow path 39 is connected to the chamber 80. The flow paths 30 to 39 may be each constituted by a tubular member such as a resin tube or a metal or glass pipe.

As illustrated in FIG. 2, the valve 40 is located among the inflow path 20, the inflow path 21, and the flow path 30. The valve 40 includes a connection port connected to the inflow path 20, a connection port connected to the inflow path 21, and a connection port connected to the flow path 30. The valve 40 may be constituted by a valve such as an electromagnetically driven valve, a piezoelectrically driven valve, or a motor-driven valve.

The valve 40 as illustrated in FIG. 2 switches the connection state among the inflow path 20, the inflow path 21, and the flow path 30 under the control of the control unit 94 as illustrated in FIG. 3. For example, the valve 40 switches the connection state among them to a state in which the inflow path 20 and the flow path 30 are connected to each other, a state in which the inflow path 21 and the flow path 30 are connected to each other, or a state in which the inflow path 20, the inflow path 21, and the flow path 30 are not connected to each other.

As illustrated in FIG. 2, the valve 41 is located among the flow path 31, the flow path 32, and the flow path 35. The valve 41 includes a connection port connected to the flow path 31, a connection port connected to the flow path 32, and a connection port connected to the flow path 35. The valve 41 may be constituted by a valve such as an electromagnetically driven valve, a piezoelectrically driven valve, or a motor-driven valve.

The valve 41 as illustrated in FIG. 2 switches the connection state among the flow path 31, the flow path 32, and the flow path 35 under the control of the control unit 94 as illustrated in FIG. 3. For example, the valve 41 switches the connection state among them to a state in which the flow path 31 and the flow path 32 are connected to each other, a state in which the flow path 35 and the flow path 32 are connected to each other, or a state in which the flow path 31, the flow path 32, and the flow path 35 are not connected to each other.

As illustrated in FIG. 2, the valve 42 is located between the flow path 33 and the flow path 34. The valve 42 includes a connection port connected to the flow path 33, and a connection port connected to the flow path 34. The valve 42 may be constituted by a valve such as an electromagnetically driven valve, a piezoelectrically driven valve, or a motor-driven valve.

The valve 42 as illustrated in FIG. 2 switches the connection state between the flow path 33 and the flow path 34 under the control of the control unit 94 as illustrated in FIG. 3. For example, the valve 42 switches the connection state between them to a state in which the flow path 33 and the flow path 34 are connected to each other or a state in which the flow path 33 and the flow path 34 are not connected to each other.

As illustrated in FIG. 2, the valve 43 is located among the discharge path 23, the flow path 36, and the flow path 37. The valve 43 includes a connection port connected to the discharge path 23, a connection port connected to the flow path 36, and a connection port connected to the flow path 37. The valve 43 may be constituted by a valve such as an electromagnetically driven valve, a piezoelectrically driven valve, or a motor-driven valve.

The valve 43 as illustrated in FIG. 2 switches the connection state among the discharge path 23, the flow path 36, and the flow path 37 under the control of the control unit 94 as illustrated in FIG. 3. For example, the valve 43 switches the connection state among them to a state in which the discharge path 23 and the flow path 36 are connected to each other, a state in which the flow path 36 and the flow path 37 are connected to each other, or a state in which the discharge path 23, the flow path 36, and the flow path 37 are not connected to each other.

As illustrated in FIG. 2, the valve 44 is located among the discharge path 24, the flow path 35, the flow path 38, and the flow path 39. The valve 44 includes a connection port connected to the discharge path 24, a connection port connected to the flow path 35, a connection port connected to the flow path 38, and a connection port connected to the flow path 39. The valve 44 may be constituted by a valve such as an electromagnetically driven valve, a piezoelectrically driven valve, or a motor-driven valve.

The valve 44 as illustrated in FIG. 2 switches the connection state among the discharge path 24, the flow path 35, the flow path 38, and the flow path 39 under the control of the control unit 94 as illustrated in FIG. 3. For example, the valve 44 switches the connection state among them to a state in which the discharge path 24 and the flow path 38 are connected to each other, a state in which the flow path 38 and the flow path 39 are connected to each other, or a state in which the flow path 35 and the flow path 38 are connected to each other. Alternatively, the valve 44 switches the connection state to a state in which the discharge path 24, the flow path 35, the flow path 38, and the flow path 39 are not connected to each other.

As illustrated in FIG. 2, the supply unit 50 is attached to the flow path 30. The supply unit 50 is capable of supplying the sample gas from the inflow path 20 to the concentration tank 60 under the control of the control unit 94 as illustrated in FIG. 3. Further, the supply unit 50 is capable of supplying the purge gas from the inflow path 21 to the storage tank 70 under the control of the control unit 94 as illustrated in FIG. 3. The arrow illustrated in the supply unit 50 indicates the direction in which the supply unit 50 sends a gas. The supply unit 50 may be constituted by a pump such as a piezoelectric pump or a motor pump. However, the supply unit 50 may be constituted by any component capable of supplying the sample gas from the inflow path 20 to the concentration tank 60.

As illustrated in FIG. 2, the inlet portion of the concentration tank 60 is connected to the flow path 32. The outlet portion of the concentration tank 60 is connected to the flow path 36. The concentration tank 60 is supplied with the sample gas flowing in from the inflow path 20 through the flow paths 30, 31, and 32. In the concentration tank 60, the sample gas is concentrated by processing described below. In this embodiment, the term “concentrating the sample gas” refers to increasing the concentration of a gas to be detected contained in the sample gas. An example of the gas to be detected will be described below. The sample gas concentrated in the concentration tank 60 is supplied to the chamber 80 through the flow paths 36 and 37.

The concentration tank 60 may be formed by a container or the like having a rectangular parallelepiped shape, a cylindrical shape, a bag shape, or a shape such that it fits in a gap between various components housed inside the housing 10. The concentration tank 60 includes an adsorbent 61, support members 62 and 63, and heaters 64.

As illustrated in FIG. 2, the adsorbent 61 is placed in the concentration tank 60. The adsorbent 61 may contain any material corresponding to the use of the gas detection system 1. The adsorbent 61 may contain, for example, at least any one of activated carbon, silica gel, zeolite, or molecular sieve. The adsorbent 61 may be of a plurality of types or may contain a porous material.

The adsorbent 61 adsorbs the gas to be detected contained in the sample gas. When the sample gas is a gas generated from feces, examples of the gas to be detected include methane, hydrogen, carbon dioxide, methyl mercaptan, hydrogen sulfide, acetic acid, and trimethylamine. The gas to be detected is, for example, a gas species that is contained in the odor of feces and is not contained in substances other than feces (such as flush water and urine) present in the toilet bowl 2A. When the sample gas is a gas generated from feces, examples of the adsorbent 61 include activated carbon and molecular sieve. However, the combination of them may be appropriately changed according to the polarity of gas molecules to be adsorbed.

In response to the adsorbent 61 reaching a predetermined temperature by being heated by the heaters 64, the gas to be detected, which is adsorbed by the adsorbent 61, can be desorbed from the adsorbent 61. The desorption of the gas to be detected from the adsorbent 61 increases the concentration of the gas to be detected in the concentration tank 60. That is, the sample gas is concentrated. Typically, a gas can be desorbed from the adsorbent 61 within a predetermined temperature range. In this embodiment, the term “desorption temperature of a gas” refers to a temperature at which the amount of the gas desorbed from the adsorbent 61 reaches a peak within a predetermined temperature range in which the gas can be desorbed from the adsorbent 61.

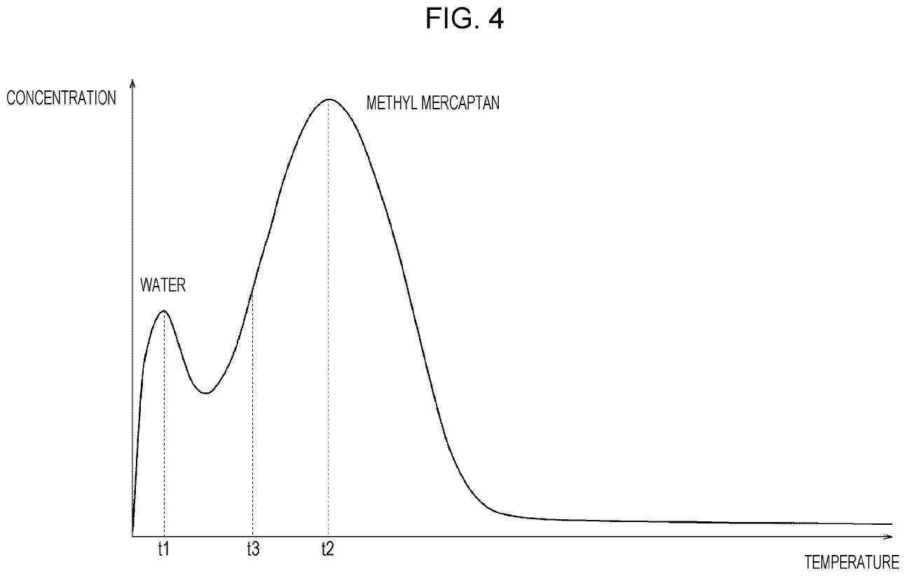

FIG. 4 is a schematic graph of the concentration of a gas desorbed from the adsorbent 61 adsorbing a predetermined gas, which is detected with a change in the temperature of the adsorbent 61. In FIG. 4, the horizontal axis represents temperature. In FIG. 4, the vertical axis represents the concentration of the gas desorbed from the adsorbent 61. The predetermined gas includes methyl mercaptan and water. Water can be desorbed from the adsorbent 61 in a predetermined temperature range including a temperature t1. The concentration (amount) of water desorbed from the adsorbent 61 reaches a peak at the temperature t1. Thus, the desorption temperature of water is the temperature t1. Methyl mercaptan can be desorbed from the adsorbent 61 in a predetermined temperature range including a temperature t2. The concentration (amount) of methyl mercaptan desorbed from the adsorbent 61 reaches a peak at the temperature t2. Thus, the desorption temperature of methyl mercaptan is the temperature t2.

The adsorbent 61 as illustrated in FIG. 2 may adsorb a gas not to be detected contained in the sample gas. The gas not to be detected is also referred to as “noise gas”. When the sample gas is a gas generated from feces, examples of the gas not to be detected include ammonia and water. A gas may have a different desorption temperature depending on the type of the gas. Accordingly, the desorption temperature of the gas to be detected and the desorption temperature of the gas not to be detected may be different. For example, in FIG. 4, when the sample gas is a gas generated from feces, the gas to be detected is methyl mercaptan. Also, the gas not to be detected is water. As illustrated in FIG. 4, the temperature t1, which is the desorption temperature of water, is different from the temperature t2, which is the desorption temperature of methylcaptan. In this embodiment, the difference in desorption temperature between gases depending on the types of the gases is utilized to exclude the gas not to be detected contained in the sample gas from the sample gas by processing described below. The gas not to be detected, which is excluded from the sample gas, is discharged to the outside through the discharge path 23.

The support member 62 as illustrated in FIG. 2 supports the adsorbent 61 near the inlet portion of the concentration tank 60. The support member 62 may be in powder or fiber form containing glass or fluorine resin.

The support member 63 as illustrated in FIG. 2 supports the adsorbent 61 near the outlet portion of the concentration tank 60. The support member 63 may be in powder or fiber form containing glass or fluorine resin.

The heaters 64 as illustrated in FIG. 2 are capable of heating the adsorbent 61. For example, the heaters 64 are energized under the control of the control unit 94 as illustrated in FIG. 3 to heat the adsorbent 61. The heaters 64 are disposed outside the concentration tank 60. The heaters 64 may surround the outer sides of the concentration tank 60. The heaters 64 may be resistance heaters, rubber heaters, or the like.

As illustrated in FIG. 2, the inlet portion of the storage tank 70 is connected to the flow path 34. The outlet portion of the storage tank 70 is connected to the flow path 38. The storage tank 70 is supplied with the purge gas flowing in from the inflow path 21 through the flow paths 30, 33, and 34. The storage tank 70 stores the supplied purge gas. The purge gas stored in the storage tank 70 is supplied to the chamber 80 through the flow paths 38 and 39. The purge gas stored in the storage tank 70 is further supplied to the concentration tank 60 through the flow paths 38, 35, and 32.

The storage tank 70 may be formed by a container or the like having a rectangular parallelepiped shape, a cylindrical shape, a bag shape, or a shape such that it fits in a gap between various components housed inside the housing 10. The storage tank 70 may have a larger capacity than the concentration tank 60. The storage tank 70 includes an adsorbent 71 and support members 72 and 73.

As illustrated in FIG. 2, the adsorbent 71 is placed in the storage tank 70. The adsorbent 71 may contain any material corresponding to the use of the gas detection system 1. The adsorbent 71 may contain, for example, at least any one of activated carbon, silica gel, zeolite, or molecular sieve. The adsorbent 71 may be of a plurality of types or may contain a porous material.

The adsorbent 71 may include an agent that adsorbs a gas to be detected contained in the purge gas. When the air in the toilet room is a purge gas, the purge gas may contain a gas to be detected. Since the adsorbent 71 adsorbs the gas to be detected contained in the purge gas, the purge gas in the storage tank 70 can be purified. When the sample gas is a gas generated from feces, examples of the adsorbent 71 that adsorbs the gas to be detected include activated carbon and molecular sieve. However, the combination of them may be appropriately changed according to the polarity of gas molecules to be adsorbed.

The adsorbent 71 may include an agent that adsorbs a gas not to be detected contained in the purge gas. When the air in the toilet room is a purge gas, the purge gas may contain a gas not to be detected. Since the adsorbent 71 adsorbs the gas not to be detected contained in the purge gas, the purge gas in the storage tank 70 can be purified. When the sample gas is a gas generated from feces, examples of the adsorbent 71 that adsorbs the gas not to be detected include silica gel and zeolite. However, the combination of them may be appropriately changed according to the polarity of gas molecules to be adsorbed.

The support member 72 supports the adsorbent 71 near the inlet portion of the storage tank 70. The support member 72 may be in powder or fiber form containing glass or fluorine resin.

The support member 73 supports the adsorbent 71 near the outlet portion of the storage tank 70. The support member 73 may be in powder or fiber form containing glass or fluorine resin.

As illustrated in FIG. 2, the chamber 80 includes therein a sensor unit 81. The chamber 80 may include a plurality of sensor units 81. The chamber 80 may be divided into a plurality of chambers. The sensor units 81 may be disposed in the resulting plurality of chambers 80. The plurality of chambers 80 may be connected to each other. The chamber 80 is connected to the flow path 37. The chamber 80 is supplied with the sample gas from the flow path 37. The chamber 80 is further connected to the flow path 39. The chamber 80 is supplied with the purge gas from the flow path 39. The chamber 80 is further connected to the discharge path 22. The chamber 80 discharges the sample gas and the purge gas, which have been subjected to detection processing, from the discharge path 22.

As illustrated in FIG. 2, the sensor unit 81 is arranged in the chamber 80. The sensor unit 81 outputs a signal corresponding to the concentration of a specific gas to the control unit 94. The sensor unit 81 may include any sensor such as a semiconductor sensor, a contact combustion sensor, or a solid electrolyte sensor. The sensor unit 81 will be described hereinafter as being configured to output a voltage corresponding to the concentration of the specific gas to the control unit 94 as the signal corresponding to the concentration of the specific gas. However, the signal corresponding to the specific gas, which is output from the sensor unit 81, is not limited to the voltage corresponding to the concentration of the specific gas. For example, the sensor unit 81 may output a current corresponding to the concentration of the specific gas to the control unit 94 as the signal corresponding to the concentration of the specific gas. The specific gas contains a specific gas to be detected and a specific gas not to be detected. When the sample gas is a gas generated from feces, examples of the specific gas to be detected include methane, hydrogen, carbon dioxide, methyl mercaptan, hydrogen sulfide, acetic acid, and trimethylamine. When the sample gas is a gas generated from feces, examples of the specific gas not to be detected include ammonia and water. Each of the plurality of sensor units 81 can output a voltage corresponding to the concentration of at least any one of these gases to the control unit 94.

The circuit board 90 as illustrated in FIG. 3 has mounted therein wiring through which an electrical signal propagates, the storage unit 91, the communication unit 92, the control unit 94, and the like.

The storage unit 91 as illustrated in FIG. 3 is constituted by, for example, a semiconductor memory, a magnetic memory, or the like. The storage unit 91 stores various kinds of information and a program for operating the gas detection system 1. The storage unit 91 may function as a work memory.

The communication unit 92 as illustrated in FIG. 3 is capable of communicating with the electronic device 3 as illustrated in FIG. 1. The communication unit 92 may be capable of communicating with an external server. The communication method used when the communication unit 92 communicates with the electronic device 3 and the external server may be a short-range wireless communication standard, a wireless communication standard for connecting to a mobile phone network, or a wired communication standard. The short-range wireless communication standard may include, for example, WiFi (registered trademark), Bluetooth (registered trademark), infrared, NFC (Near Field Communication), and the like. The wireless communication standard for connecting to a mobile phone network may include, for example, LTE (Long Term Evolution), a fourth generation or higher mobile communication system, or the like. Alternatively, the communication method used when the communication unit 92 communicates with the electronic device 3 and the external server may be, for example, a communication standard such as LPWA (Low Power Wide Area) or LPWAN (Low Power Wide Area Network).

The sensor unit 93 as illustrated in FIG. 3 may include at least any one of an image camera, a personal identification switch, an infrared sensor, a pressure sensor, or the like. The sensor unit 93 outputs a detection result to the control unit 94.

For example, when the sensor unit 93 includes an infrared sensor, the sensor unit 93 detects reflected light from an object irradiated with infrared radiation from the infrared sensor, thereby being able to detect that the subject has entered the toilet room. The sensor unit 93 outputs, as a detection result, a signal indicating that the subject has entered the toilet room to the control unit 94.

For example, when the sensor unit 93 includes a pressure sensor, the sensor unit 93 detects a pressure applied to the toilet seat 2B as illustrated in FIG. 1, thereby being able to detect that the subject has sat on the toilet seat 2B. The sensor unit 93 outputs, as a detection result, a signal indicating that the subject has sat on the toilet seat 2B to the control unit 94.

For example, when the sensor unit 93 includes a pressure sensor, the sensor unit 93 detects a reduction in the pressure applied to the toilet seat 2B as illustrated in FIG. 1, thereby being able to detect that the subject has risen from the toilet seat 2B. The sensor unit 93 outputs, as a detection result, a signal indicating that the subject has risen from the toilet seat 2B to the control unit 94.

For example, when the sensor unit 93 includes an image camera, a personal identification switch, and the like, the sensor unit 93 collects data, such as a face image, the sitting height, and the weight. The sensor unit 93 identifies and detects a person from the collected data. The sensor unit 93 outputs, as a detection result, a signal indicating the identified person to the control unit 94.

For example, when the sensor unit 93 includes a personal identification switch or the like, the sensor unit 93 identifies (detects) a person in response to an operation of the personal identification switch. In this case, personal information may be registered (stored) in the storage unit 91 in advance. The sensor unit 93 outputs, as a detection result, a signal indicating the identified person to the control unit 94.

The control unit 94 as illustrated in FIG. 3 includes one or more processors. The one or more processors may include at least any one of a general-purpose processor that reads a specific program to execute a specific function, or a dedicated processor dedicated to a specific process. The dedicated processor may include an application specific IC (ASIC; Application Specific Integrated Circuit). The one or more processors may include a programmable logic device (PLD). The PLD may include an FPGA. (Field-Programmable Gate Array). The control unit 94 may include at least any one of an SoC (System-on-a-chip) or an SiP (System-in-a-Package) with which the one or more processors cooperate.

<Purge Gas Storage Process>

The control unit 94 can detect that the subject has risen from the toilet seat 2B on the basis of the detection result of the sensor unit 93. The control unit 94 performs control so that the air in the toilet room flows into the inflow path 21 as a purge gas after a predetermined first set time period has elapsed since it was detected that the subject rose from the toilet seat 2B. The control unit 94 performs control so that the purge gas flowing in from the inflow path 21 is stored in the storage tank 70. The first set time period may be appropriately set in consideration of the time period taken to replace the air in the toilet room with air outside the toilet room by using a ventilation fan or the like in the toilet room after the subject exits the toilet room.

For example, the control unit 94 causes the valve 40 as illustrated in FIG. 2 to connect the inflow path 21 and the flow path 30 to each other, and causes the valve 42 as illustrated in FIG. 2 to connect the flow path 33 and the flow path 34 to each other. Further, the control unit 94 causes the valve 44 as illustrated in FIG. 2 to connect the flow path 38 and the discharge path 24 to each other. In addition, the control unit 94 controls the supply unit 50 to generate a flow of gas from the inflow path 21 toward the discharge path 24 through the flow paths 30, 33, and 34, the storage tank 70, and the flow path 38. As a result of generation of the flow of gas, the air in the toilet room flows into the inflow path 21 as a purge gas. The purge gas flowing in from the inflow path 21 is supplied to the storage tank 70 through the flow paths 30, 33, and 34. Since the purge gas is supplied to the storage tank 70, the residual gas in the storage tank 70 is pushed out to the flow path 38 by the purge gas and discharged from the discharge path 24. The control unit 94 stops the supply unit 50 at a point in time when a predetermined second set time period elapses after the purge gas starts to flow into the inflow path 21. Further, the control unit 94 causes the valve 40 not to connect the inflow path 21 and the flow path 30 to each other, and causes the valve 42 not to connect the flow path 33 and the flow path 34 to each other. In addition, the control unit 94 causes the valve 44 not to connect the flow path 38 and the discharge path 24 to each other. With this configuration, the purge gas from the inflow path 21 is stored in the storage tank 70. The second set time period may be appropriately set in consideration of the capacity of the storage tank 70 and the like. The purge gas stored in the storage tank 70 can come into contact with the adsorbent 71 in the storage tank 70. Since the purge gas comes into contact with the adsorbent 71, the gas to be detected and the gas not to be detected contained in the purge gas can be adsorbed by the adsorbent 71. Since the gas to be detected and the gas not to be detected contained in the purge gas are adsorbed by the adsorbent 71, the purge gas in the storage tank 70 can be purified.

<Sample Gas Storage and Concentration Process>

The control unit 94 as illustrated in FIG. 3 can detect that the subject has sat on the toilet seat 2B on the basis of the detection result of the sensor unit 93. The control unit 94 performs control so that a gas generated from feces discharged into the toilet bowl 2A flows into the inflow path 20 as a sample gas after a predetermined third set time period has elapsed since it was detected that the subject sat on the toilet seat 2B. The control unit 94 performs control so that the sample gas flowing in from the inflow path 20 passes through the concentration tank 60. The third set time period may be appropriately set in consideration of the time period taken until the subject defecates after the subject sits on the toilet seat 2B.

For example, the control unit 94 causes the valve 40 as illustrated in FIG. 2 to connect the inflow path 20 and the flow path 30 to each other, and causes the valve 41 to connect the flow path 31 and the flow path 32 to each other. Further, the control unit 94 causes the valve 43 as illustrated in FIG. 2 to connect the flow path 36 and the discharge path 23 to each other. In addition, the control unit 94 controls the supply unit 50 as illustrated in FIG. 2 to generate a flow of gas from the inflow path 20 toward the discharge path 23 through the flow paths 30, 31, and 32, the concentration tank 60, and the flow path 36. As a result of generation of the flow of gas, the sample gas flowing in from the inflow path 20 passes through the concentration tank 60.

The control unit 94 as illustrated in FIG. 3 performs control so that the sample gas passes through the concentration tank 60 to cause the adsorbent 61 to adsorb a detection target gas contained in the sample gas. For example, the control unit 94 may perform control so that the sample gas passes through the concentration tank 60 for a predetermined first time period. The first time period may be appropriately set in consideration of the amount of the gas to be detected that can be adsorbed by the adsorbent 61. The control unit 94 may further control the supply unit 50 so that the flow rate of the sample gas passing through the inside of the concentration tank 60 is a first flow rate. The first flow rate may be appropriately set in consideration of the volumetric capacity of the concentration tank 60, the area of the adsorbent 61, or the like. Further, the control unit 94 may maintain the heaters 64 in a non-driven state while the sample gas passes through the inside of the concentration tank 60. Since the heaters 64 are maintained in the non-driven state, the temperature of the adsorbent 61 can be room temperature. The control unit 94 may estimate the flow rate of the sample gas from at least any one of a driving voltage, a frequency, or the like of a pump or the like constituting the supply unit 50. The gas detection system 1 may be provided with a flow rate sensor that detects the flow rate of the sample gas. In this configuration, the flow rate sensor outputs a detection signal indicating the flow rate of the sample gas to the control unit 94. The control unit 94 detects the flow rate of the sample gas on the basis of the detection signal output from the flow rate sensor. The control unit 94 may also detect the flow rate of the purge gas in a manner that is the same as or similar to that of the sample gas.

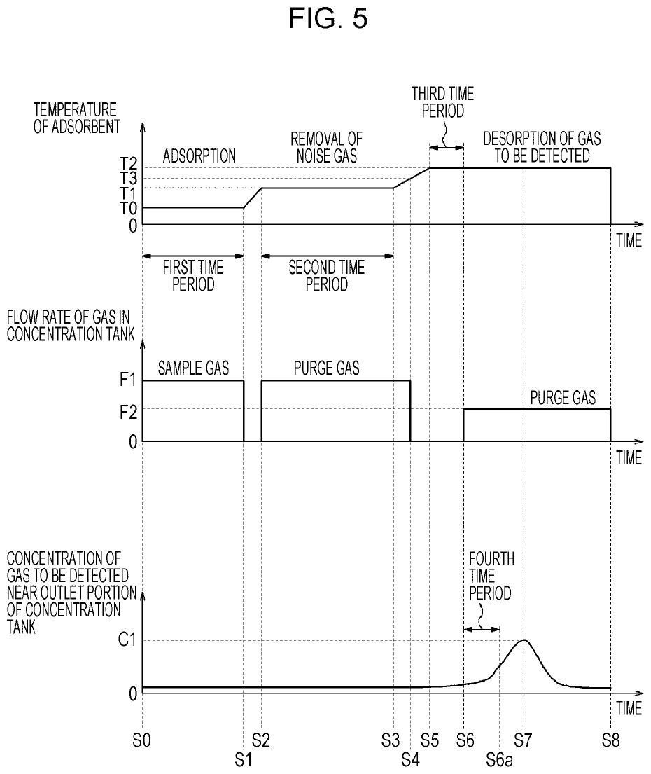

FIG. 5 is a timing chart of an example operation of the gas detection system 1 illustrated in FIG. 1. The upper part of FIG. 5 illustrates a change in the temperature of the adsorbent 61 with time. The central part of FIG. 5 illustrates changes in the flow rates of gases in the concentration tank 60 with time. The lower part of FIG. 5 illustrates a change in the concentration of the gas to be detected near the outlet portion of the concentration tank 60 with time. The control unit 94 may estimate the temperature of the adsorbent 61 from the current of the heaters 64 or the like. A temperature sensor may be disposed in the vicinity of the adsorbent 61. In this configuration, the temperature sensor outputs a signal indicating the temperature in the vicinity of the adsorbent 61 to the control unit 94. The control unit 94 may acquire the temperature of the adsorbent 61 on the basis of the detection signal output from the temperature sensor.

Time 50 as illustrated in FIG. 5 is a point in time at which the third set time period elapses after the control unit 94 detects that the subject has sat on the toilet seat 2B. At the time S0, the control unit 94 performs control so that a gas generated from feces discharged into the toilet bowl 2A flows into the inflow path 20 as a sample gas. The control unit 94 further performs control so that the sample gas passes through the concentration tank 60. In this case, the control unit 94 controls the supply unit 50 so that the flow rate of the sample gas passing through the concentration tank 60 is a first flow rate F1. Further, the control unit 94 maintains the heaters 64 in the non-driven state. Since the heaters 64 are maintained in the non-driven state, the adsorbent 61 is maintained at room temperature T0. The control unit 94 performs control so that the sample gas passes through the concentration tank 60 for the first time period from the time S0 to time S1.

At the time S0 as illustrated in FIG. 5, the sample gas starts to pass through the concentration tank 60. Since the sample gas starts to pass through the concentration tank 60, the adsorbent 61 starts to adsorb the gas to be detected contained in the sample gas. The sample gas in which the gas to be detected is adsorbed by the adsorbent 61 while passing through the concentration tank 60 is discharged from the discharge path 23. If the sample gas contains a gas not to be detected, the gas not to be detected can also be adsorbed by the adsorbent 61 after the time S0.

The control unit 94 as illustrated in FIG. 3 stops the passage of the sample gas to the concentration tank 60 at a point in time when the first time period elapses after the sample gas starts to pass through the concentration tank 60. For example, the control unit 94 stops the supply unit 50 at a point in time when the first time period elapses. Further, the control unit 94 causes the valve 41 not to connect the flow path 31 and the flow path 32 to each other, and causes the valve 43 not to connect the flow path 36 and the discharge path 23 to each other. At the point in time when the first time period elapses, the control unit 94 brings the heaters 64 into the driven state to increase the temperature of the adsorbent 61.

The time S1 as illustrated in FIG. 5 is the point in time when the first time period elapses after the sample gas starts to pass through the concentration tank 60. At the time S1, the control unit 94 stops the passage of the sample gas to the concentration tank 60. Stopping the passage of the sample gas to the concentration tank 60 reduces the flow rate of the gas in the concentration tank 60 to 0. At the time S1, furthermore, the control unit 94 brings the heaters 64 into the driven state. Since the heaters 64 are brought into the driven state at the time S1, the temperature of the adsorbent 61 increases after the time S1.

In response to the temperature of the adsorbent 61 as illustrated in FIG. 2 reaching a temperature T1, for example, the control unit 94 as illustrated in FIG. 3 performs control so that the temperature of the adsorbent 61 is maintained as the temperature T1 for a predetermined second time period. The second time period may be appropriately set in consideration of the amount of the gas not to be detected that can be contained in the sample gas. The temperature T1 may be the desorption temperature of the gas not to be detected that can be contained in the sample gas. For example, when the gas not to be detected is water, the temperature T1 can be the temperature t1 as illustrated in FIG. 4. Since the adsorbent 61 is maintained at the temperature T1, the gas not to be detected can be desorbed from the adsorbent 61. The control unit 94 performs control so that the purge gas passes through the concentration tank 60 while performing control so that the temperature of the adsorbent 61 is maintained as the temperature T1. The control unit 94 performs control so that the purge gas that has passed through the concentration tank 60 is discharged from the discharge path 23. With this configuration, the gas not to be detected desorbed from the adsorbent 61 can be discharged from the discharge path 23 together with the purge gas. That is, the gas not to be detected desorbed from the adsorbent 61 can be removed from the concentration tank 60. The control unit 94 may control the supply unit 50 so that the flow rate of the purge gas passing through the inside of the concentration tank 60 is the first flow rate.

For example, in response to the temperature of the adsorbent 61 as illustrated in FIG. 2 reaching the temperature T1, the control unit 94 causes the valve 40 to connect the inflow path 21 and the flow path 30 to each other, and causes the valve 42 to connect the flow path 33 and the flow path 34 to each other. The control unit 94 further causes the valve 44 to connect the flow path 38 and the flow path 35 to each other, causes the valve 41 to connect the flow path 35 and the flow path 32 to each other, and causes the valve 43 to connect the flow path 36 and the discharge path 23 to each other. In addition, the control unit 94 controls the supply unit 50 to generate a flow of gas from the inflow path 21 toward the discharge path 23 through the flow paths 30, 33, and 34, the storage tank 70, and the flow paths 38, 35, and 32, the concentration tank 60, and the flow path 36. As a result of generation of the flow of the gas, the purge gas passes through the concentration tank 60 and is discharged from the discharge path 23. Since the purge gas passes through the concentration tank 60, the gas not to be detected desorbed from the adsorbent 61 is removed from the concentration tank 60 and discharged from the discharge path 23.

At time S2 as illustrated in FIG. 5, the temperature of the adsorbent 61 reaches the temperature T1. The control unit 94 controls the heaters 64 so that the temperature of the adsorbent 61 is maintained as the temperature T1 for the second time period from the time S2 to time S3. Since the temperature of the adsorbent 61 is maintained as the temperature T1 after the time S2, the gas not to be detected can be desorbed from the adsorbent 61. Further, the control unit 94 performs control so that the purge gas passes through the concentration tank 60 and is discharged from the discharge path 23 at the time S2. Since the purge gas passes through the concentration tank 60 and is discharged from the discharge path 23, the gas not to be detected desorbed from the adsorbent 61 can be removed from the concentration tank 60 and discharged from the discharge path 23. The control unit 94 may control the supply unit 50 so that the flow rate of the purge gas passing through the inside of the concentration tank 60 is the first flow rate F1.

At a point in time when the second time period elapses after the temperature of the adsorbent 61 as illustrated in FIG. 2 reaches the temperature T1, the control unit 94 as illustrated in FIG. 3 controls the heaters 64 so that the temperature of the adsorbent 61 increases. The control unit 94 controls the heaters 64 so that the temperature of the adsorbent 61 increases to a temperature T2. The temperature T2 may be the desorption temperature of the gas to be detected contained in the sample gas. For example, when the gas to be detected is methyl mercaptan, the temperature T2 can be the temperature t2 as illustrated in FIG. 4. In response to the temperature of the adsorbent 61 reaching the temperature T2, the control unit 94 controls the heaters 64 so that the temperature of the adsorbent 61 is maintained as the temperature T2.

The time S3 as illustrated in FIG. 5 is a point in time at which the second time period elapses. At the time S3, the control unit 94 controls the heaters 64 so that the temperature of the adsorbent 61 increases. At time S5, the temperature of the adsorbent 61 reaches the temperature T2. The control unit 94 performs control so that the temperature of the adsorbent 61 is maintained as the temperature T2 after the time S5.

In the present disclosure, the control unit 94 as illustrated in FIG. 3 stops the passage of the purge gas to the concentration tank 60 from a first point in time to a second point in time later than the first point in time. The first point in time in the present disclosure is a point in time before or at which the temperature of the adsorbent 61 reaches the temperature T2. In the first embodiment, the first point in time is a point in time at which the temperature of the adsorbent 61 reaches a temperature T3. The temperature T3 may be set on the basis of a temperature at which the gas to be detected starts to be desorbed from the adsorbent 61. For example, when the gas to be detected is methyl mercaptan, the temperature T3 can be a temperature t3 as illustrated in FIG. 4. In the first embodiment, the second point in time is a point in time at which, for example, a predetermined third time period elapses after the temperature of the adsorbent 61 reaches the temperature T2. In response to the temperature of the adsorbent 61 reaching the temperature T3, the gas to be detected starts to be desorbed from the adsorbent 61 inside the concentration tank 60. As a result of stopping the passage of the purge gas to the concentration tank 60 for a time period from the first point in time to the second point in time, a large amount of the gas to be detected that have been desorbed from the adsorbent 61 can remain in the concentration tank 60. Since a large amount of the gas to be detected remains in the concentration tank 60, the concentration of the gas to be detected in the concentration tank 60 can be increased. That is, the sample gas can further be concentrated in the concentration tank 60. The third time period may be appropriately set in consideration of the amount of detection target gas that can be adsorbed by the adsorbent 61.

For example, the control unit 94 stops the supply unit 50 in response to the temperature of the adsorbent 61 as illustrated in FIG. 2 reaching the temperature T3. Further, the control unit 94 causes the valve 41 not to connect the flow path 31, the flow path 32, and the flow path 35 to each other, and causes the valve 43 not to connect the discharge path 23, the flow path 36, and the flow path 37 to each other. With this configuration, the passage of the purge gas to the concentration tank 60 is stopped.

The control unit 94 as illustrated in FIG. 3 performs control so that the purge gas passes through the concentration tank 60 after the second point in time. In this embodiment, the control unit 94 performs control so that the purge gas passes through the concentration tank 60 from the second point in time, that is, from the point in time at which the third time period elapses after the temperature of the adsorbent 61 reaches the temperature T2. The control unit 94 performs control so that the purge gas passes through the concentration tank 60 and is supplied to the sensor unit 81 in the chamber 80 together with the gas to be detected in the concentration tank 60. With this configuration, the gas to be detected having an increased concentration in the concentration tank 60, that is, the more concentrated sample gas in the concentration tank 60, can be transported to the sensor unit 81 in the chamber 80 by the purge gas. The purge gas is also referred to as a “carrier gas” when used in gas transportation applications.

For example, at the second point in time, the control unit 94 causes the valve 40 as illustrated in FIG. 2 to connect the inflow path 21 and the flow path 30 to each other, and causes the valve 42 to connect the flow path 33 and the flow path 34 to each other. The control unit 94 further causes the valve 44 to connect the flow path 38 and the flow path 35 to each other, causes the valve 41 to connect the flow path 35 and the flow path 32 to each other, and causes the valve 43 to connect the flow path 36 and the flow path 37 to each other. In addition, the control unit 94 controls the supply unit 50 to generate a flow of gas from the inflow path 21 toward the chamber 80 through the flow paths 30, 33, and 34, the storage tank 70, and the flow paths 38, 35, and 32, the concentration tank 60, and the flow paths 36 and 37. As a result of generation of the flow of gas, the purge gas passes through the concentration tank 60 and transports the gas to be detected in the concentration tank 60 to the chamber 80.

At time S4 as illustrated in FIG. 5, the temperature of the adsorbent 61 reaches the temperature T3. That is, the time S4 corresponds to the first point in time. At the time S5, the temperature of the adsorbent 61 reaches the temperature T2. The time period from the time S5 to time S6 is the third time period. That is, the time S6 corresponds to the second point in time. The control unit 94 stops the passage of the purge gas to the concentration tank 60 from the time S4 to the time S6. At the time S6, the control unit 94 performs control so that the purge gas passes through the concentration tank 60 and is supplied to the chamber 80 together with the gas to be detected in the concentration tank 60. The control unit 94 may perform control so that the purge gas passes through the concentration tank 60 for a time period from the time S6 to time S8. The time period from the time S6 to the time S8 may be appropriately set in accordance with to the volumetric capacity of the storage tank 70 and the like.

The purge gas supplied from the inlet portion of the concentration tank 60 can gradually push out the gas to be detected in the concentration tank 60 toward the outlet portion of the concentration tank 60. Accordingly, the concentration of the gas to be detected in the vicinity of the outlet portion in the concentration tank 60 may become maximum after a certain time period has elapsed since the start of supply of the purge gas to the concentration tank 60, depending on the type of the gas to be detected. In the example as illustrated in FIG. 5, at time S7, the concentration of the gas to be detected in the vicinity of the outlet portion in the concentration tank 60 becomes a maximum value C1. That is, at the beginning when the purge gas starts to pass through the concentration tank 60, the concentration of the detection target gas that is transported by the purge gas may not be so high.

Accordingly, the control unit 94 may exhaust the purge gas that has passed through the concentration tank 60 from, for example, the discharge path 23 without supplying the purge gas to the sensor unit 81 until a predetermined fourth time period elapses after the purge gas starts to pass through the concentration tank 60. Further, the control unit 94 may supply the purge gas that has passed through the concentration tank 60 to the sensor unit 81 after the fourth time period has elapsed. In this case, the control unit 94 may cause the valve 43 to connect the flow path 36 and the discharge path 23 to each other until the fourth time period elapses after the purge gas starts to pass through the concentration tank 60, thereby discharging the purge gas that has passed through the concentration tank 60 from the discharge path 23. Further, after the fourth time period has elapsed, the control unit 94 may cause the valve 43 to connect the flow path 36 and the flow path 37 to each other to supply the purge gas that has passed through the concentration tank 60 to the sensor unit 81. The fourth time period may be appropriately set in consideration of the length, cross-sectional area, and the like of the concentration tank 60. The fourth time period may be about a time period from the time S6 to time S6a as illustrated in FIG. 5. The time S6a is a time later than the time S6 and is a time immediately before the time S7. Further, the control unit 94 may control the supply unit 50 so that the flow rate of the purge gas passing through the inside of the concentration tank 60 is the second flow rate. In FIG. 5, the control unit 94 may control the supply unit 50 so that the flow rate of the purge gas is a second flow rate F2 after the time S6. The second flow rate is smaller than the first flow rate. The second flow rate may be appropriately determined in consideration of the volumetric capacity of the concentration tank 60, the specifications of the valve 43, and the like. Since the flow rate of the purge gas passing through the inside of the concentration tank 60 is set to the second flow rate smaller than the first flow rate, the control unit 94 can cause the valve 43 to smoothly switch the connection destination of the flow path 36 from the discharge path 23 to the flow path 37 after the lapse of the fourth time period.

<Process for Detecting Type and Concentration of Gas>

The control unit 94 performs control so that the purge gas stored in the storage tank 70 is supplied to the sensor unit 81 in the chamber 80. For example, the control unit 94 causes the valve 40 to connect the inflow path 21 and the flow path 30 to each other, causes the valve 42 to connect the flow path 33 and the flow path 34 to each other, and causes the valve 44 to connect the flow path 38 and the flow path 39 to each other. Further, the control unit 94 controls the supply unit 50 to generate a flow of gas from the inflow path 21 toward the chamber 80 through the flow paths 30, 33, and 34, the storage tank 70, and the flow paths 38 and 39. As a result of generation of the flow of gas, the purge gas stored in the storage tank 70 is supplied to the sensor unit 81 in the chamber 80.

The control unit 94 performs control so that the purge gas passes through the concentration tank 60 and is supplied to the sensor unit 81 in the chamber 80 together with the gas to be detected in the concentration tank 60 in the way described in the <Sample Gas Storage and Concentration Process> section described above.

The control unit 94 performs control so that the purge gas stored in the storage tank 70 and the sample gas concentrated in the concentration tank 60 are alternately supplied to the sensor unit 81 in the chamber 80. The control unit 94 alternately supplies the purge gas and the concentrated sample gas to the chamber 80 to acquire a voltage waveform from the sensor unit 81 in the chamber 80. The control unit 94 detects the type and concentration of a gas contained in the sample gas by, for example, machine learning for the acquired voltage waveform. The control unit 94 may transmit the detected type and concentration of the gas to the electronic device 3 via the communication unit 92 as a detection result.

[Operation of Gas Detection System]

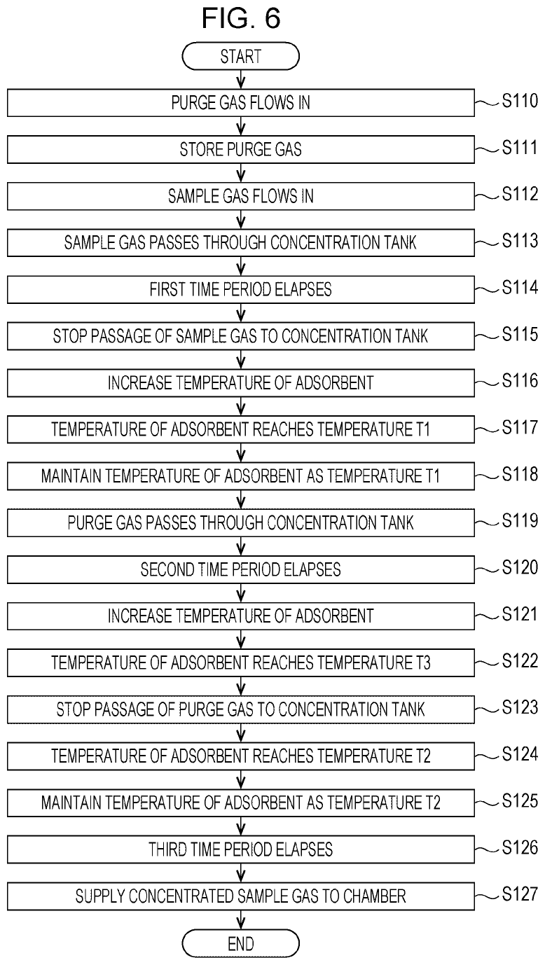

FIG. 6 is a flowchart of an example operation of the gas detection system 1 illustrated in FIG. 1 during gas concentration. The control unit 94 may start a process as illustrated in FIG. 6 after the first set time period elapses after it is detected that the subject has risen from the toilet seat 2B on the basis of the detection result of the sensor unit 93.

The control unit 94 performs control so that the air in the toilet room flows into the inflow path 21 as a purge gas (step S110). The control unit 94 performs control so that the purge gas flowing into the inflow path 21 is stored in the storage tank 70 (step S111).

The control unit 94 performs control so that a gas generated from feces discharged into the toilet bowl 2A flows into the inflow path 20 as a sample gas after the third set time period has elapsed since it was detected that the subject sat on the toilet seat 2B (step S112). The control unit 94 performs control so that the sample gas flowing in from the inflow path 20 passes through the concentration tank 60 for the first time period (step S113).

The control unit 94 detects the lapse of the first time period after the sample gas starts to pass through the concentration tank 60 (step S114). The control unit 94 stops the passage of the sample gas to the concentration tank 60 at the point in time at which the first time period elapses (step S115). The control unit 94 controls the heaters 64 so that the temperature of the adsorbent 61 increases (step S116).

The control unit 94 detects the temperature of the adsorbent 61 reaching the temperature T1 (step S117). In response to the temperature of the adsorbent 61 reaching the temperature T1, the control unit 94 controls the heaters 64 so that the temperature of the adsorbent 61 is maintained as the temperature T1 for the second time period (step S118). The control unit 94 performs control so that the purge gas passes through the concentration tank 60 while performing control so that the temperature of the adsorbent 61 is maintained as the temperature T1 (step S119).

The control unit 94 detects the lapse of the second time period after the temperature of the adsorbent 61 reaches the temperature T1 (step S120). The control unit 94 controls the heaters 64 so that the temperature of the adsorbent 61 increases at the point in time at which the second time period elapses (step S121).

The control unit 94 detects the temperature of the adsorbent 61 reaching the temperature T3 (step S122). In response to the temperature of the adsorbent 61 reaching the temperature T3, the control unit 94 stops the passage of the purge gas to the concentration tank 60 (step S123).

The control unit 94 detects the temperature of the adsorbent 61 reaching the temperature T2 (step S124). The control unit 94 controls the heaters 64 so that the temperature of the adsorbent 61 is maintained as the temperature T2 (step S125).

The control unit 94 detects the lapse of the third time period after the temperature of the adsorbent 61 reaches the temperature T2 (step S135). The control unit 94 performs control so that the purge gas passes through the concentration tank 60 and is supplied to the sensor unit 81 in the chamber 80 together with the gas to be detected in the concentration tank 60 at the point in time at which the third time period elapses (step S127).

In the processing of step S113, the control unit 94 may control the supply unit 50 so that the flow rate of the sample gas passing through the inside of the concentration tank 60 is the first flow rate. In the processing of step S117, the control unit 94 may control the supply unit 50 so that the flow rate of the purge gas passing through the inside of the concentration tank 60 is the first flow rate.

In the processing of step S127, the control unit 94 may exhaust the purge gas that has passed through the concentration tank 60 from, for example, the discharge path 23 without supplying the purge gas to the sensor unit 81 until the fourth time period elapses after the purge gas starts to pass through the concentration tank 60. Further, the control unit 94 may supply the purge gas that has passed through the concentration tank 60 to the sensor unit 81 after the fourth time period has elapsed. In this case, the control unit 94 may control the supply unit 50 so that the flow rate of the purge gas passing through the inside of the concentration tank 60 is the second flow rate. After the processing of step S127 ends, the control unit 94 ends the gas concentration process.

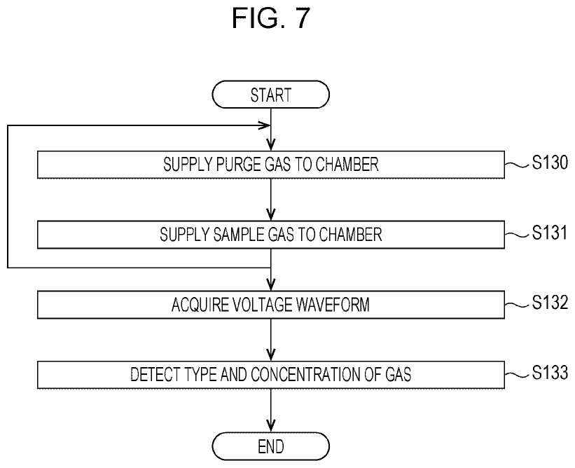

FIG. 7 is a flowchart of an example operation of the gas detection system 1 illustrated in FIG. 1 during detection of the type and concentration of a gas.

The control unit 94 performs control so that the purge gas stored in the storage tank 70 is supplied to the sensor unit 81 in the chamber 80 (step S130). The control unit 94 executes the process as illustrated in FIG. 6 to perform control so that the purge gas passes through the concentration tank 60 and is supplied to the sensor unit 81 in the chamber 80 together with the gas to be detected in the concentration tank 60 (step S131).

The control unit 94 alternately executes the processing of step S130 and the processing of step S131 to perform control so that the purge gas in the storage tank 70 and the sample gas in the concentration tank 60 are alternately supplied to the sensor unit 81 in the chamber 80.

The control unit 94 alternately supplies the purge gas and the concentrated sample gas to the chamber 80 to acquire a voltage waveform from the sensor unit 81 in the chamber 80 (step S132). The control unit 94 detects the type and concentration of a gas contained in the sample gas by, for example, machine learning for the acquired voltage waveform (step S133). After the processing of step S133 ends, the control unit 94 ends the process for detecting the type and concentration of the gas.

As described above, in the gas detection system 1 according to the first embodiment, the control unit 94 stops the passage of the purge gas to the concentration tank 60 from the first point in time to the second point in time. Further, the control unit 94 performs control so that the purge gas passes through the concentration tank 60 and is supplied to the sensor unit 81 in the chamber 80 from the second point in time. With this configuration, the more concentrated sample gas in the concentration tank 60 can be supplied to the sensor unit 81 in the chamber 80 by the purge gas. Since the more concentrated sample gas is supplied to the sensor unit 81, the gas to be detected contained in the sample gas can be more reflected in the voltage output from the sensor unit 81. Since the gas to be detected is more reflected in the voltage output from the sensor unit 81, the gas detection system 1 can more accurately detect the type and concentration of the gas contained in the sample gas. Accordingly, this embodiment can provide the gas detection system 1 with improved gas detection performance and the like.

### Second Embodiment

A gas detection system according to a second embodiment of the present disclosure will be described hereinafter. The gas detection system according to the second embodiment can adopt a configuration that is the same as or similar to that of the gas detection system 1 according to the first embodiment. The following mainly describes differences from the first embodiment with reference to FIGS. 1 to 3.

As described above in the first embodiment, the control unit 94 as illustrated in FIG. 3 controls the supply unit 50 so that the sample gas passes through the concentration tank 60 as illustrated in FIG. 2. In this case, the control unit 94 controls the supply unit 50 so that the flow rate of the sample gas passing through the concentration tank 60 is the first flow rate in a manner that is the same as or similar to that in the first embodiment.

As described above in the first embodiment, the control unit 94 as illustrated in FIG. 3 controls the supply unit 50 so that the purge gas passes through the concentration tank 60 while controlling the heaters 64 to increase the temperature of the adsorbent 61 as illustrated in FIG. 2. In this case, the control unit 94 controls the supply unit 50 so that the flow rate of the purge gas passing through the inside of the concentration tank 60 is the first flow rate in a manner that is the same as or similar to that in the first embodiment.

As described above in the first embodiment, the control unit 94 as illustrated in FIG. 3 stops the passage of the purge gas to the concentration tank 60 from the first point in time to the second point in time, and, from the second point in time, performs control so that the purge gas passes through the concentration tank 60 and is supplied to the sensor unit 81. In this case, in the second embodiment, the control unit 94 controls the supply unit 50 so that the flow rate of the purge gas passing through the inside of the concentration tank 60 is a third flow rate. The third flow rate is larger than the first flow rate. The third flow rate may be appropriately determined in consideration of the volumetric capacity of the concentration tank 60, a desired detection time period in the gas detection system 1, and the like. Since the flow rate of the purge gas passing through the inside of the concentration tank 60 is set to the third flow rate larger than the first flow rate, the time taken to supply the sample gas in the concentration tank 60 to the chamber 80 by the purge gas can be shortened. With this configuration, the detection time period in the gas detection system 1 can be shortened.

In the following, the first point in time according to the second embodiment will be described as a point in time at which the temperature of the adsorbent 61 reaches the temperature T3 in a manner that is the same as or similar to that in the first embodiment. The second point in time according to the second embodiment will be described as a point in time at which the third time period elapses after the temperature of the adsorbent 61 reaches the temperature T2 in a manner that is the same as or similar to that in the first embodiment. The third time period according to the second embodiment may be appropriately set in consideration of the amount of detection target gas that can be adsorbed by the adsorbent 61 in a manner that is the same as or similar to that in the first embodiment. However, the third time period according to the second embodiment may be set independently of the third time period according to the first embodiment. For example, as described above, when the flow rate of the purge gas passing through the inside of the concentration tank 60 is set to the third flow rate, the time taken to supply the sample gas in the concentration tank 60 to the chamber 80 by the purge gas can be shortened. The third time period may be increased as the time taken to supply the sample gas to the chamber 80 is reduced. Increasing the third time period can increase the time for stopping the passage of the purge gas to the concentration tank 60. Since the time for stopping the passage of the purge gas to the concentration tank 60 is increased, a larger amount of gas to be detected can be desorbed from the adsorbent 61 for a period during which the passage of the purge gas to the concentration tank 60 is stopped. That is, the sample gas can further be concentrated in the concentration tank 60. Accordingly, a more concentrated sample gas can be supplied to the sensor unit 81.

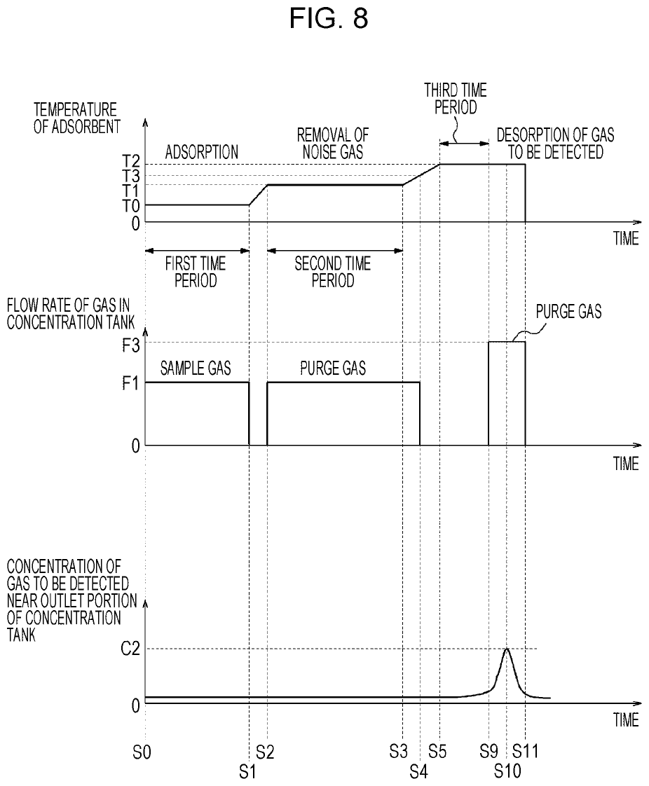

FIG. 8 is timing chart of an example operation of the gas detection system 1 according to the second embodiment of the present disclosure. The upper part of FIG. 8 illustrates a change in the temperature of the adsorbent 61 with time. The central part of FIG. 8 illustrates changes in the flow rates of gases in the concentration tank 60 with time. The lower part of FIG. 8 illustrates a change in the concentration of the gas to be detected near the outlet portion of the concentration tank 60 with time.

From time S0 to time S5 as illustrated in FIG. 8, the control unit 94 performs control in a manner that is the same as or similar to that from the time S0 to the time S5 as illustrated in FIG. 5. At time S4 as illustrated in FIG. 8, the temperature of the adsorbent 61 reaches the temperature T3 in a manner that is the same as or similar to that in FIG. 5. That is, the time S4 as illustrated in FIG. 8 corresponds to the first point in time in a manner that is the same as or similar to that in FIG. 5. At the time S5 as illustrated in FIG. 8, the temperature of the adsorbent 61 reaches the temperature T2 in a manner that is the same as or similar to that in FIG. 5.

In FIG. 8, a time period from the time S5 to time S9 is the third time period. That is, the time S9 corresponds to the second point in time. The control unit 94 stops the passage of the purge gas to the concentration tank 60 from the time S4 to the time S9. At the time S9, the control unit 94 performs control so that the purge gas passes through the concentration tank 60 and is supplied to the chamber 80 together with the gas to be detected in the concentration tank 60. The control unit 94 performs control so that the flow rate of the purge gas is a third flow rate F3. At time S10, the concentration of the gas to be detected in the vicinity of the outlet portion in the concentration tank 60 becomes a maximum value C2. The control unit 94 may perform control so that the purge gas passes through the concentration tank 60 for a time period from the time S9 to time S11. The time period from the time S9 to the time S11 may be appropriately set on the basis of the third flow rate F3.

As described above, in the gas detection system 1 according to the second embodiment, when performing control so that the purge gas passes through the concentration tank 60 from the second point in time, the control unit 94 performs control so that the flow rate of the purge gas passing through the inside of the concentration tank 60 is the third flow rate. The third flow rate is larger than the first flow rate. With this configuration, in the second embodiment, the detection time period in the gas detection system 1 can be shortened.

Other configurations and advantages of the gas detection system 1 according to the second embodiment are the same as or similar or to those of the gas detection system 1 according to the first embodiment.

(Modifications of First Embodiment and Second Embodiment)

Modifications of the first embodiment and the second embodiment will be described hereinafter.

For example, in the first embodiment described above, as illustrated in FIG. 5, the control unit 94 has been described as being configured to cause the purge gas to pass through the concentration tank 60 and to be supplied to the sensor unit 81 in the chamber 80 for the time period from the time S6 to the time S8. However, the control of the control unit 94 is not limited to this. For example, the control unit 94 may alternately supply the sample gas in the concentration tank 60 and the purge gas in the storage tank 70 to the sensor unit 81 for the time period from the time S6 to the time S8. In this case, the control unit 94 may detect the type and concentration of a gas contained in the sample gas on the basis of the voltage output from the sensor unit 81 around the time S7 at which the concentration of the gas to be detected is the maximum value C1. This applies to the second embodiment in the same or similar manner.

For example, in the first embodiment and the second embodiment described above, the control unit 94 has been described as being configured to perform control so that the purge gas passes through the concentration tank 60 and is supplied to the sensor unit 81 in the chamber 80 together with the gas to be detected in the concentration tank 60 from the second point in time. However, in the present disclosure, the control unit 94 performs control so that the purge gas passes through the concentration tank 60 and is supplied to the sensor unit 81 in the chamber 80 together with the gas to be detected in the concentration tank 60 after the second point in time. For example, the gas detection system 1 may include a buffer tank between the concentration tank 60 and the chamber 80. In this case, the control unit 94 may perform control so that the purge gas passes through the concentration tank 60 and is stored in the buffer tank from the second point in time. Further, after performing control so that the purge gas passes through the concentration tank 60 and is stored in the buffer tank, the control unit 94 may stop the supply of the purge gas to the concentration tank 60. Since the supply of the purge gas to the concentration tank 60 is stopped, the gas to be detected desorbed from the adsorbent 61 can have uniform concentration in the buffer tank. In addition, after stopping the supply of the purge gas to the concentration tank 60, the control unit 94 may perform control so that the sample gas stored in the buffer tank is supplied to the sensor unit 81 in the chamber 80. With this configuration, the sample gas in which the gas to be detected has uniform concentration can be supplied to the sensor unit 81 in the chamber 80.

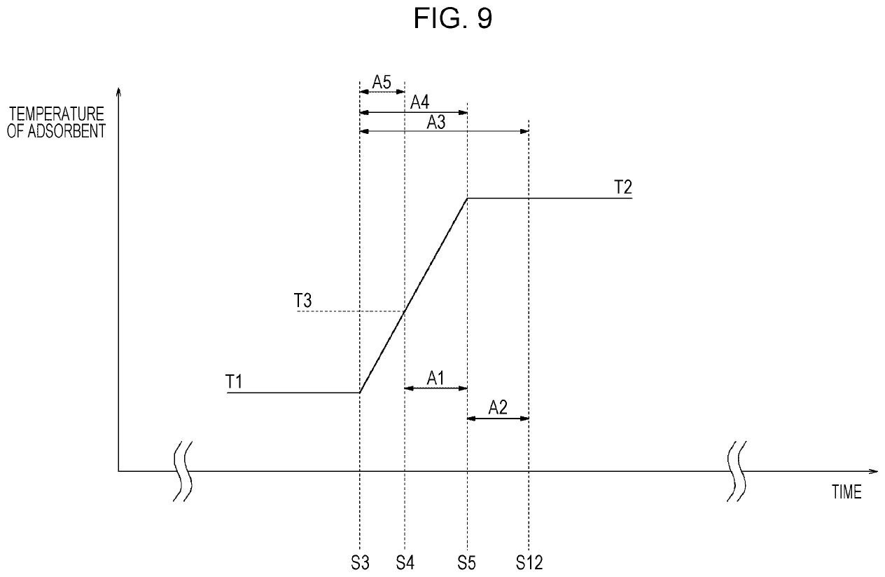

For example, in the first embodiment and the second embodiment, the first point in time has been described as a point in time at which the temperature of the adsorbent 61 reaches the temperature T3. However, the first point in time is not limited to this. As described above, the first point in time in the present disclosure is a point in time before the temperature of the adsorbent 61 reaches the temperature T2 or a point in time at which the temperature of the adsorbent 61 reaches the temperature T2. In the first embodiment and the second embodiment described above, furthermore, the second point in time has been described as a point in time at which the third time period elapses after the temperature of the adsorbent 61 reaches the temperature T2. However, the second point in time is not limited to this. As described above, the second point in time in the present disclosure is a point in time later than the first point in time. Another example of the first point in time and the second point in time will be described with reference to FIG. 9.

FIG. 9 is a timing chart describing another example of the first point in time and the second point in time in the present disclosure. At time S3, the control unit 94 controls the heaters 64 so that the temperature of the adsorbent 61 increases in a manner that is the same as or similar to that in FIG. 5. At time S4, the temperature of the adsorbent 61 reaches the temperature T3 in a manner that is the same as or similar to that in FIG. 5. At time S5, the temperature of the adsorbent 61 reaches the temperature T2 in a manner that is the same as or similar to that in FIG. 5.

For example, the first point in time may be a point in time at which the temperature T3 is reached. That is, the time S4 as illustrated in FIG. 9 may correspond to the first point in time. The second point in time may be a point in time at which the temperature of the adsorbent 61 reaches the desorption temperature of the gas to be detected. That is, the time S5 may correspond to the second point in time. In this case, the control unit 94 stops the passage of the purge gas to the concentration tank 60 for a time period A1 from the time S4 to the time S5 as illustrated in FIG. 9. Further, the control unit 94 performs control so that the purge gas passes through the concentration tank 60 and is supplied to the sensor unit 81 in the chamber 80 from the time S5 as illustrated in FIG. 9. With this configuration, the time period during which the passage of the purge gas to the concentration tank 60 is stopped can be shorter than that in a case where the purge gas is stopped from the time S4 to the time S6 as illustrated in FIG. 5, for example. Since the time period during which the passage of the purge gas is stopped is short, the amount of the gas to be detected that is not desorbed from the adsorbent 61 at the time S5 as illustrated in FIG. 9, which is the second point in time, can be larger than the amount of the gas to be detected that is not desorbed from the adsorbent 61 at the time S6 as illustrated in FIG. 5, which is the second point in time. Since the amount of the gas to be detected that is not desorbed from the adsorbent 61 at the second point in time is large, the gas to be detected can be desorbed from the adsorbent 61 even while the purge gas is controlled to pass through the concentration tank 60 from the second point in time. With this configuration, the sample gas in which the gas to be detected has a relatively high concentration can be continuously supplied to the sensor unit 81 for a longer period of time.

For example, the first point in time may be a point in time at which the temperature of the adsorbent 61 reaches the temperature T2. That is, the time S5 as illustrated in FIG. 9 may correspond to the first point in time. The second point in time may be a point in time at which the third time period elapses after the temperature of the adsorbent 61 reaches the temperature T2. A time period from the time S5 to a time S12 as illustrated in FIG. 9 may be the third time period. That is, the time S12 as illustrated in FIG. 9 may correspond to the second point in time. In this case, the control unit 94 stops the passage of the purge gas to the concentration tank 60 for a time period A2 from the time S5 to the time S12 as illustrated in FIG. 9. Further, the control unit 94 performs control so that the purge gas passes through the concentration tank 60 and is supplied to the sensor unit 81 in the chamber 80 from the time S12 as illustrated in FIG. 9. With this configuration, the passage of the purge gas to the concentration tank 60 can be stopped after the temperature of the adsorbent 61 reaches the temperature T2. Since the passage of the purge gas to the concentration tank 60 is stopped after the temperature of the adsorbent 61 reaches the temperature T2, the probability that the gas not to be detected is desorbed from the adsorbent 61 can be reduced while the passage of the purge gas is stopped. Since the probability that the gas not to be detected is desorbed from the adsorbent 61 is reduced, the concentration of the gas to be detected can be increased.

For example, the first point in time may be a point in time at which the temperature of the adsorbent 61 exceeds the temperature T1, which is the desorption temperature of the detection target gas. That is, the time S3 as illustrated in FIG. 9 may correspond to the first point in time. The second point in time may be a point in time at which the third time period elapses after the temperature of the adsorbent 61 reaches the temperature T2. The time period from the time S5 to the time S12 as illustrated in FIG. 9 may be the third time period. That is, the time S12 as illustrated in FIG. 9 may correspond to the second point in time. In this case, the control unit 94 stops the passage of the purge gas to the concentration tank 60 for a time period A3 from the time S3 to the time S12 as illustrated in FIG. 9. Further, the control unit 94 performs control so that the purge gas passes through the concentration tank 60 and is supplied to the sensor unit 81 in the chamber 80 from the time S12 as illustrated in FIG. 9.

For example, the first point in time may be a point in time at which the temperature of the adsorbent 61 exceeds the temperature T1, which is the desorption temperature of the detection target gas. That is, the time S3 as illustrated in FIG. 9 may correspond to the first point in time. The second point in time may be a point in time at which the temperature of the adsorbent 61 reaches the temperature T2. That is, the time S5 as illustrated in FIG. 9 may correspond to the second point in time. In this case, the control unit 94 stops the passage of the purge gas to the concentration tank 60 for a time period A4 from the time S3 to the time S5 as illustrated in FIG. 9. Further, the control unit 94 performs control so that the purge gas passes through the concentration tank 60 and is supplied to the sensor unit 81 in the chamber 80 from the time S5 as illustrated in FIG. 9.

For example, the first point in time may be a point in time at which the temperature of the adsorbent 61 exceeds the temperature T1, which is the desorption temperature of the detection target gas. That is, the time S3 as illustrated in FIG. 9 may correspond to the first point in time. The second point in time may be a point in time at which the temperature T3 is reached. That is, the time S4 as illustrated in FIG. 9 may correspond to the second point in time. In this case, the control unit 94 stops the passage of the purge gas to the concentration tank 60 for a time period A5 from the time S3 to the time S4 as illustrated in FIG. 9. Further, the control unit 94 performs control so that the purge gas passes through the concentration tank 60 and is supplied to the sensor unit 81 in the chamber 80 from the time S4 as illustrated in FIG. 9.

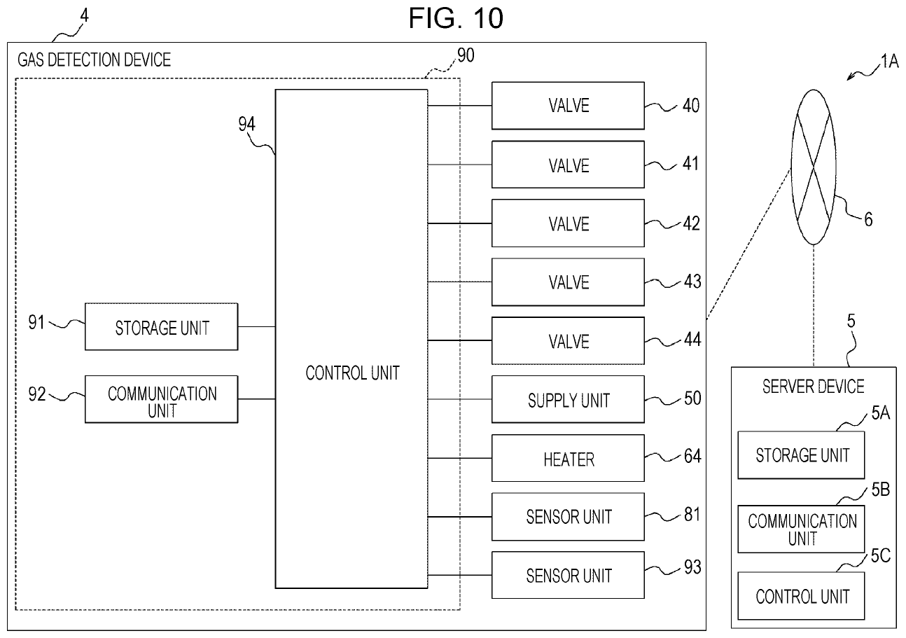

For example, in the first embodiment and the second embodiment described above, as illustrated in FIG. 3, the gas detection system 1 has been described as a single device. However, the gas detection system according to the present disclosure is not limited to the single device. The gas detection system according to the present disclosure may include a plurality of independent devices. For example, the first embodiment and the second embodiment described above may adopt a gas detection system 1A having a configuration as illustrated in FIG. 10.

As illustrated in FIG. 10, the gas detection system 1A includes a gas detection device 4 and a server device 5. The gas detection device 4 and the server device 5 are capable of communicating with each other via a network 6. A portion of the network 6 may be wired or wireless. The gas detection device 4 has a configuration that is the same as or similar to the configuration of the gas detection system 1 as illustrated in FIG. 2 and FIG. 3. The server device 5 includes a storage unit 5A, a communication unit 5B, and a control unit 5C. The control unit 5C is capable of executing the processes of the control unit 94 as illustrated in FIG. 3 described above. For example, the control unit 5C stops the passage of the purge gas to the concentration tank 60 as illustrated in FIG. 2 from the first point in time to the second point in time, and, after the second point in time, performs control so that the purge gas passes through the concentration tank 60 and is supplied to the sensor unit 81 in the chamber 80.

### Third Embodiment

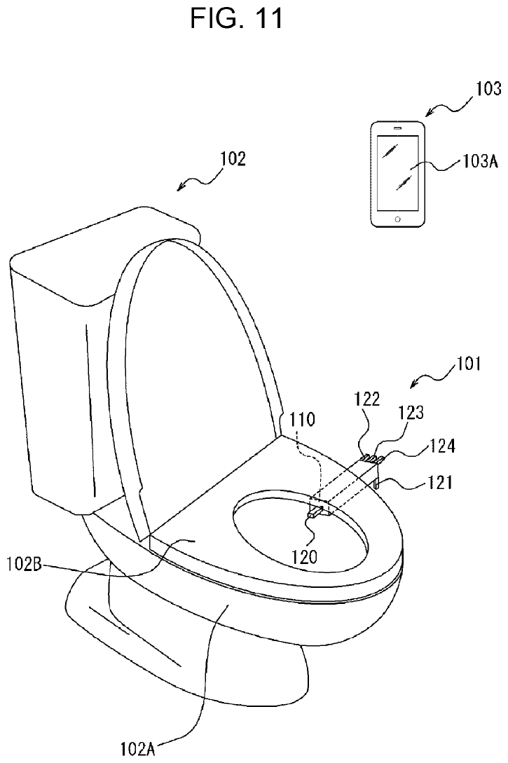

As illustrated in FIG. 11, a gas detection system 101 is installed in a toilet 102. The toilet 102 may be, but is not limited to, a flush toilet. The toilet 102 includes a toilet bowl 102A and a toilet seat 102B. The gas detection system 101 may be installed in any portion of the toilet 102. In one example, as illustrated in FIG. 11, the gas detection system 101 may be arranged from between the toilet bowl 102A and the toilet seat 102B to the outside of the toilet 102. A portion of the gas detection system 101 may be embedded inside the toilet seat 102B. The subject can discharge feces into the toilet bowl 102A. The gas detection system 101 can acquire a gas generated from the feces discharged into the toilet bowl 102A as a sample gas. The gas detection system 101 can detect the type of a gas contained in the sample gas, the concentration of the gas, and so on. The gas detection system 101 can transmit the detection results and so on to an electronic device 103. The gas detection system 101 as illustrated in FIG. 11 is also referred to as a “gas detection device”.

The uses of the gas detection system 101 are not limited to the use described above. For example, the gas detection system 101 may be installed in a refrigerator. In this case, the gas detection system 101 can acquire a gas generated from food as a sample gas. In another use, for example, the gas detection system 101 may be installed in a factory or a laboratory. In this case, the gas detection system 101 can acquire a gas generated from a chemical or the like as a sample gas.

The toilet 102 can be installed in a toilet room in a house, a hospital, or the like. The toilet 102 can be used by the subject. As described above, the toilet 102 includes the toilet bowl 102A and the toilet seat 102B. The subject can discharge feces into the toilet bowl 102A.

The electronic device 103 is, for example, a smartphone used by the subject. However, the electronic device 103 is not limited to a smartphone. The electronic device 103 may be any electronic device. When brought into the toilet room by the subject, as illustrated in FIG. 11, the electronic device 103 can be present in the toilet room. However, for example, when the subject does not bring the electronic device 103 into the toilet room, the electronic device 103 may be present outside the toilet room. The electronic device 103 can receive the detection results from the gas detection system 101 via wireless communication or wired communication. The electronic device 103 can display the received detection results on a display unit 103A. The display unit 103A may include a display capable of displaying characters and the like, and a touch screen capable of detecting contact of a finger of the user (subject) or the like. The display may include a display device such as a liquid crystal display (LCD), an organic EL display (GELD), or an inorganic EL display (IELD). The detection method of the touch screen may be any method such as a capacitance method, a resistance film method, a surface acoustic wave method, an ultrasonic method, an infrared method, an electromagnetic induction method, or a load detection method.

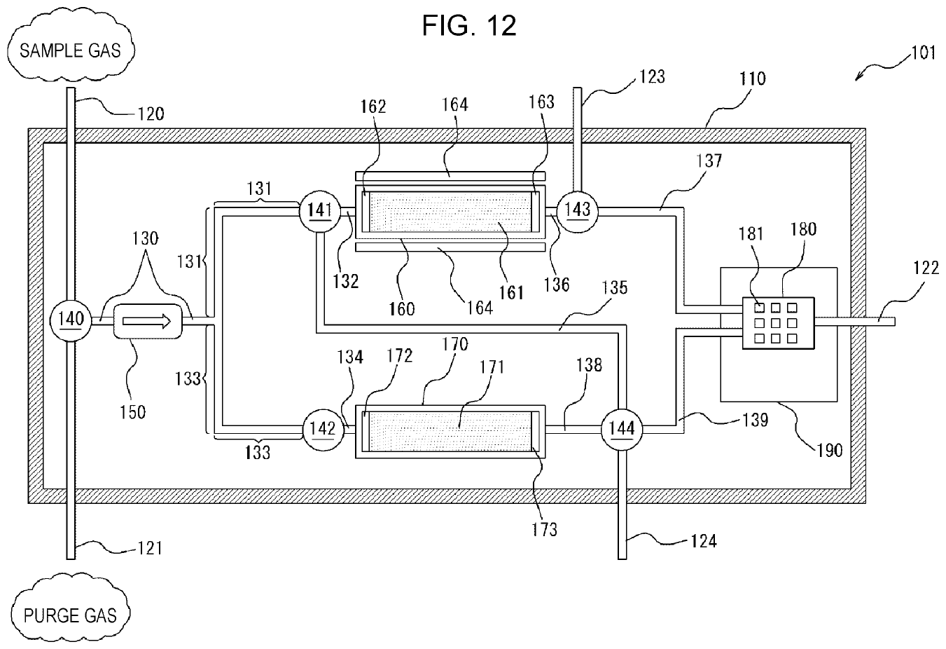

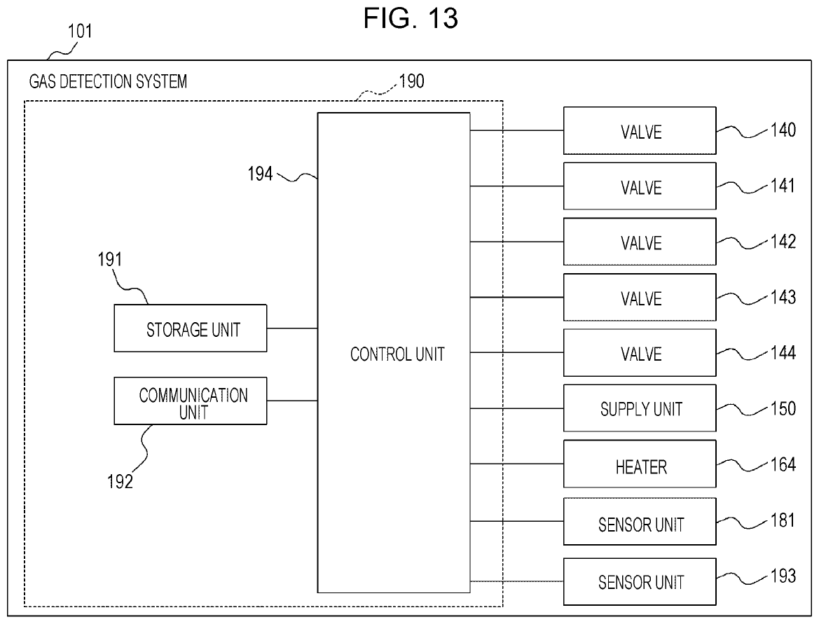

As illustrated in FIG. 12, the gas detection system 101 includes a housing 110, inflow paths 120 and 121, and discharge paths 122, 123, and 124. The discharge path 122, the discharge path 123, and the discharge path 124 may merge in any location. The gas detection system 101 includes flow paths 130, 131, 132, 133, 134, 135, 136, 137, 138, and 139, valves 140, 141, 142, 143, and 144, and a supply unit 150. The gas detection system 101 includes a concentration tank 160 serving as a gas concentration unit, a storage tank 170 serving as a gas reservoir, a chamber 180, and a circuit board 190 serving as a circuit unit. As illustrated in FIG. 13, the gas detection system 101 includes, in the circuit board 190, a storage unit 191, a communication unit 192, and a control unit 194. The gas detection system 101 includes a sensor unit 193.

The housing 110 houses various components of the gas detection system 101. The housing 110 may be made of any material. For example, the housing 110 may be made of a material such as metal or resin.

As illustrated in FIG. 11, the inflow path 120 can be exposed to the inside of the toilet bowl 102A. A portion of the inflow path 120 may be embedded in the toilet seat 102B. A gas generated from feces discharged into the toilet bowl 102A flows into the inflow path 120 as a sample gas. The sample gas flowing into the inflow path 120 is supplied to the concentration tank 160 through the flow paths 130, 131, and 132. As illustrated in FIG. 11, one end of the inflow path 120 may be directed to the inside of the toilet bowl 102A. As illustrated in FIG. 12, the other end of the inflow path 120 may be connected to the valve 140. The inflow path 120 may be constituted by a tubular member such as a resin tube or a metal or glass pipe.

As illustrated in FIG. 11, the inflow path 121 can be exposed to the outside of the toilet bowl 102A. A portion of the inflow path 121 may be embedded in the toilet seat 102B. For example, air in the toilet room, which is outside the toilet bowl 102A, flows into the inflow path 121 as a purge gas. The purge gas flowing into the inflow path 121 is supplied to the storage tank 170 through the flow paths 130, 133, and 134. As illustrated in FIG. 11, one end of the inflow path 121 may be directed to the outside of the toilet 102. As illustrated in FIG. 12, the other end of the inflow path 121 may be connected to the valve 140. The inflow path 121 may be constituted by a tubular member such as a resin tube or a metal or glass pipe.

As illustrated in FIG. 11, a portion of the discharge path 122 can be exposed to the outside of the toilet bowl 102A. The discharge path 122 as illustrated in FIG. 12 discharges the exhaust from the chamber 180 to the outside. This exhaust can contain the sample gas and the purge gas, which have been subjected to detection processing. As illustrated in FIG. 11, one end of the discharge path 122 may be directed to the outside of the toilet 102. As illustrated in FIG. 12, the other end of the discharge path 122 may be connected to the chamber 180. The discharge path 122 may be constituted by a tubular member such as a resin tube or a metal or glass pipe.

As illustrated in FIG. 11, a portion of the discharge path 123 can be exposed to the outside of the toilet bowl 102A. The discharge path 123 as illustrated in FIG. 12 discharges the exhaust from the concentration tank 160 to the outside. This exhaust includes a gas not to be detected, which is generated in a concentration process of the sample gas described below. As illustrated in FIG. 11, one end of the discharge path 123 may be directed to the outside of the toilet 102. As illustrated in FIG. 12, the other end of the discharge path 123 may be connected to the valve 143. The discharge path 123 may be constituted by a tubular member such as a resin tube or a metal or glass pipe.

As illustrated in FIG. 11, a portion of the discharge path 124 can be exposed to the outside of the toilet bowl 102A. The discharge path 124 as illustrated in FIG. 12 discharges the residual gas or the like from the storage tank 170 to the outside. As illustrated in FIG. 11, one end of the discharge path 124 may be directed to the outside of the toilet 102. As illustrated in FIG. 12, the other end of the discharge path 124 may be connected to the valve 144. The discharge path 124 may be constituted by a tubular member such as a resin tube or a metal or glass pipe.

As illustrated in FIG. 12, one end of the flow path 130 is connected to the valve 140. The other end of the flow path 130 is connected to one end of the flow path 131 and one end of the flow path 133. The one end of the flow path 131 is connected to the other end of the flow path 130. The other end of the flow path 131 is connected to the valve 141. One end of the flow path 132 is connected to the valve 141. The other end of the flow path 132 is connected to an inlet portion of the concentration tank 160. The one end of the flow path 133 is connected to the other end of the flow path 130. The other end of the flow path 133 is connected to the valve 142. One end of the flow path 134 is connected to the valve 142. The other end of the flow path 134 is connected to an inlet portion of the storage tank 170. One end of the flow path 135 is connected to the valve 141. The other end of the flow path 135 is connected to the valve 144. One end of the flow path 136 is connected to an outlet portion of the concentration tank 160. The other end of the flow path 136 is connected to the valve 143. One end of the flow path 137 is connected to the valve 143. The other end of the flow path 137 is connected to the chamber 180. One end of the flow path 138 is connected to an outlet portion of the storage tank 170. The other end of the flow path 138 is connected to the valve 144. One end of the flow path 139 is connected to the valve 144. The other end of the flow path 139 is connected to the chamber 180. The flow paths 130 to 139 may be each constituted by a tubular member such as a resin tube or a metal or glass pipe.

As illustrated in FIG. 12, the valve 140 is located among the inflow path 120, the inflow path 121, and the flow path 130. The valve 140 includes a connection port connected to the inflow path 120, a connection port connected to the inflow path 121, and a connection port connected to the flow path 130. The valve 140 may be constituted by a valve such as an electromagnetically driven valve, a piezoelectrically driven valve, or a motor-driven valve.

The valve 140 as illustrated in FIG. 12 switches the connection state among the inflow path 120, the inflow path 121, and the flow path 130 under the control of the control unit 194 as illustrated in FIG. 13. For example, the valve 140 switches the connection state among them to a state in which the inflow path 120 and the flow path 130 are connected to each other, a state in which the inflow path 121 and the flow path 130 are connected to each other, or a state in which the inflow path 120, the inflow path 121, and the flow path 130 are not connected to each other.

As illustrated in FIG. 12, the valve 141 is located among the flow path 131, the flow path 132, and the flow path 135. The valve 141 includes a connection port connected to the flow path 131, a connection port connected to the flow path 132, and a connection port connected to the flow path 135. The valve 141 may be constituted by a valve such as an electromagnetically driven valve, a piezoelectrically driven valve, or a motor-driven valve.

The valve 141 as illustrated in FIG. 12 switches the connection state among the flow path 131, the flow path 132, and the flow path 135 under the control of the control unit 194 as illustrated in FIG. 13. For example, the valve 141 switches the connection state among them to a state in which the flow path 131 and the flow path 132 are connected to each other, a state in which the flow path 135 and the flow path 132 are connected to each other, or a state in which the flow path 131, the flow path 132, and the flow path 135 are not connected to each other.

As illustrated in FIG. 12, the valve 142 is located between the flow path 133 and the flow path 134. The valve 142 includes a connection port connected to the flow path 133, and a connection port connected to the flow path 134. The valve 142 may be constituted by a valve such as an electromagnetically driven valve, a piezoelectrically driven valve, or a motor-driven valve.

The valve 142 as illustrated in FIG. 12 switches the connection state between the flow path 133 and the flow path 134 under the control of the control unit 194 as illustrated in FIG. 13. For example, the valve 142 switches the connection state between them to a state in which the flow path 133 and the flow path 134 are connected to each other or a state in which the flow path 133 and the flow path 134 are not connected to each other.

As illustrated in FIG. 12, the valve 143 is located among the discharge path 123, the flow path 136, and the flow path 137. The valve 143 includes a connection port connected to the discharge path 123, a connection port connected to the flow path 136, and a connection port connected to the flow path 137. The valve 143 may be constituted by a valve such as an electromagnetically driven valve, a piezoelectrically driven valve, or a motor-driven valve.

The valve 143 as illustrated in FIG. 12 switches the connection state among the discharge path 123, the flow path 136, and the flow path 137 under the control of the control unit 194 as illustrated in FIG. 13. For example, the valve 143 switches the connection state among them to a state in which the discharge path 123 and the flow path 136 are connected to each other, a state in which the flow path 136 and the flow path 137 are connected to each other, or a state in which the discharge path 123, the flow path 136, and the flow path 137 are not connected to each other.

As illustrated in FIG. 12, the valve 144 is located among the discharge path 124, the flow path 135, the flow path 138, and the flow path 139. The valve 144 includes a connection port connected to the discharge path 124, a connection port connected to the flow path 135, a connection port connected to the flow path 138, and a connection port connected to the flow path 139. The valve 144 may be constituted by a valve such as an electromagnetically driven valve, a piezoelectrically driven valve, or a motor-driven valve.

The valve 144 as illustrated in FIG. 12 switches the connection state among the discharge path 124, the flow path 135, the flow path 138, and the flow path 139 under the control of the control unit 194 as illustrated in FIG. 13. For example, the valve 144 switches the connection state among them to a state in which the discharge path 124 and the flow path 138 are connected to each other, a state in which the flow path 138 and the flow path 139 are connected to each other, or a state in which the flow path 135 and the flow path 138 are connected to each other. Alternatively, the valve 144 switches the connection state to a state in which the discharge path 124, the flow path 135, the flow path 138, and the flow path 139 are not connected to each other.

As illustrated in FIG. 12, the supply unit 150 is attached to the flow path 130. The supply unit 150 is capable of supplying the sample gas from the inflow path 120 to the concentration tank 160 under the control of the control unit 194 as illustrated in FIG. 13. Further, the supply unit 150 is capable of supplying the purge gas from the inflow path 121 to the storage tank 170 under the control of the control unit 194 as illustrated in FIG. 13. The arrow illustrated in the supply unit 150 indicates the direction in which the supply unit 150 sends a gas. The supply unit 150 may be constituted by a pump such as a piezoelectric pump or a motor pump. However, the supply unit 150 may be constituted by any component capable of supplying the sample gas from the inflow path 120 to the concentration tank 160.

As illustrated in FIG. 12, the inlet portion of the concentration tank 160 is connected to the flow path 132. The outlet portion of the concentration tank 160 is connected to the flow path 136. The concentration tank 160 is supplied with the sample gas flowing in from the inflow path 120 through the flow paths 130, 131, and 132. In the concentration tank 160, the sample gas is concentrated by processing described below. In this embodiment, the term “concentrating the sample gas” refers to increasing the concentration of a gas to be detected contained in the sample gas. An example of the gas to be detected will be described below. The sample gas concentrated in the concentration tank 160 is supplied to the chamber 180 through the flow paths 136 and 137.

The concentration tank 160 may be formed by a container or the like having a rectangular parallelepiped shape, a cylindrical shape, a bag shape, or a shape such that it fits in a gap between various components housed inside the housing 110. The concentration tank 160 includes an adsorbent 161, support members 162 and 163, and heaters 164.

As illustrated in FIG. 12, the adsorbent 161 is placed in the concentration tank 160. The adsorbent 161 may contain any material corresponding to the use of the gas detection system 1. The adsorbent 161 may contain, for example, at least any one of activated carbon, silica gel, zeolite, or molecular sieve. The adsorbent 161 may be of a plurality of types or may contain a porous material.

The adsorbent 161 adsorbs the gas to be detected contained in the sample gas. When the sample gas is a gas generated from feces, examples of the gas to be detected include methane, hydrogen, carbon dioxide, methyl mercaptan, hydrogen sulfide, acetic acid, and trimethylamine. The gas to be detected is, for example, a gas species that is contained in the odor of feces and is not contained in substances other than feces (such as flush water and urine, for example) present in the toilet bowl 102A. When the sample gas is a gas generated from feces, examples of the adsorbent 161 include activated carbon and molecular sieve. However, the combination of them may be appropriately changed according to the polarity of gas molecules to be adsorbed.

In response to the adsorbent 161 reaching a predetermined temperature by being heated by the heaters 164, the gas to be detected, which is adsorbed by the adsorbent 161, can be desorbed from the adsorbent 161. The desorption of the gas to be detected from the adsorbent 161 increases the concentration of the gas to be detected in the concentration tank 160. That is, the sample gas is concentrated. Typically, a gas can be desorbed from the adsorbent 161 within a predetermined temperature range. In this embodiment, the term “desorption temperature of a gas” refers to a temperature at which the amount of the gas desorbed from the adsorbent 161 reaches a peak within a predetermined temperature range in which the gas can be desorbed from the adsorbent 161.

The adsorbent 161 may adsorb a gas not to be detected contained in the sample gas. The gas not to be detected is also referred to as “noise gas”. When the sample gas is a gas generated from feces, examples of the gas not to be detected include ammonia and water. A gas may have a different desorption temperature depending on the type of the gas. Accordingly, the desorption temperature of the gas to be detected and the desorption temperature of the gas not to be detected may be different. In this embodiment, the difference in desorption temperature between gases depending on the types of the gases is utilized to exclude the gas not to be detected contained in the sample gas from the sample gas by processing described below. The gas not to be detected, which is excluded from the sample gas, is discharged to the outside through the discharge path 123.

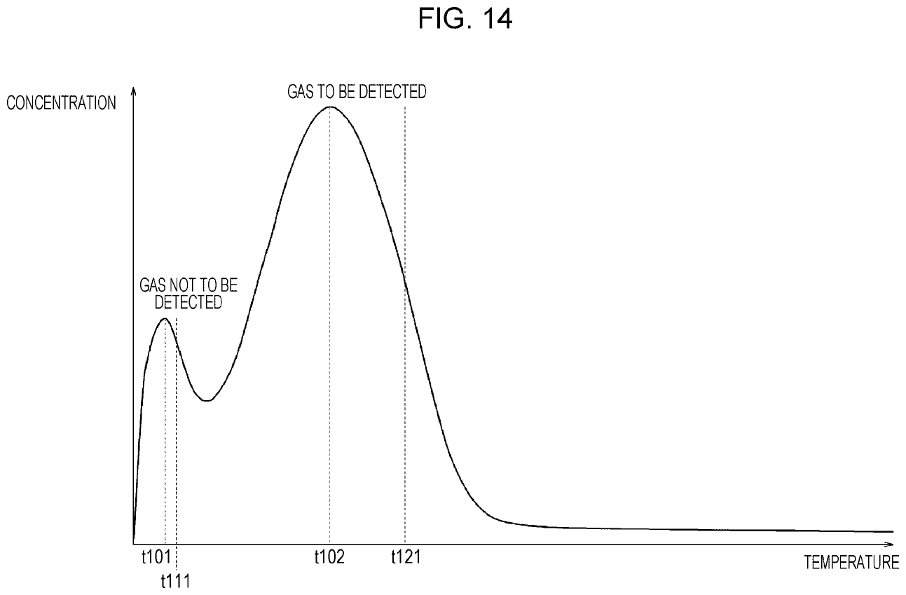

FIG. 14 is a schematic graph of the concentration of a gas desorbed from an adsorbent 161X adsorbing a predetermined gas, which is detected with a change in the temperature of the adsorbent 161X. The adsorbent 161X does not have a pore 161a described below. In FIG. 14, the horizontal axis represents temperature. In FIG. 14, the vertical axis represents the concentration of the gas desorbed from the adsorbent 161X. The predetermined gas includes a gas to be detected and a gas not to be detected. The gas not to be detected can be desorbed from the adsorbent 161X in a predetermined temperature range including a temperature t101. The concentration (amount) of the gas not to be detected desorbed from the adsorbent 161X reaches a peak at the temperature t101. Thus, the desorption temperature of the gas not to be detected is the temperature t101. The gas to be detected can be desorbed from the adsorbent 161X in a predetermined temperature range including a temperature t102. The concentration (amount) of the gas to be detected desorbed from the adsorbent 161X reaches a peak at the temperature t102. Thus, the desorption temperature of the gas to be detected is the temperature t102. As described above, the desorption temperature of the gas not to be detected, namely, the temperature t101, and the desorption temperature of the gas to be detected, namely, the temperature t102, are different. In this embodiment, the difference in desorption temperature between the gas not to be detected and the gas to be detected is utilized to exclude the gas not to be detected contained in the sample gas from the sample gas by processing described below.

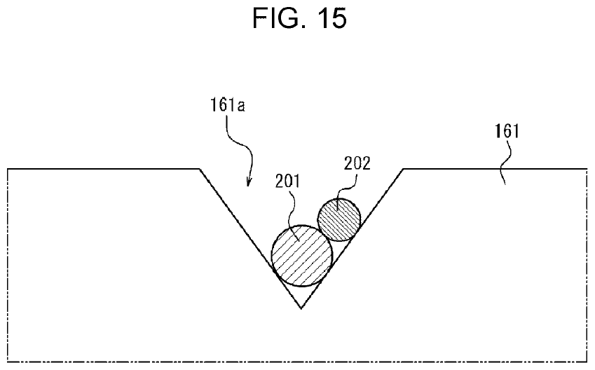

As illustrated in FIG. 15, the adsorbent 161 has a pore 161a. The pore 161a has a larger pore size than the effective molecular diameter (hereinafter, “effective molecular diameter” is also referred to simply as “molecular diameter”) of a gas to be detected 201 and the molecular diameter of a gas not to be detected 202. In the present disclosure, the term “pore size” means the average diameter value of the pore 161a of the adsorbent 161 on the surfaces of the adsorbent 161. The pore size of the pore 161a can be measured using, for example, a pore distribution measurement apparatus. In this case, the average value of the pore size can be calculated as, for example, the average pore size (4 V/A). That is, the average pore size can be determined from the specific surface area (A) and the total pore volume (V). The specific surface area can be determined by using a BET one point method. The total pore volume can be determined by using a one point method total pore volume. Since the specific surface area and the total pore volume are determined in this way, the average pore size can be easily determined. Alternatively, the pore size of the pore 161a can be easily measured by image analysis using a scanning electron microscope.

Since the pore size of the pore 161a is larger than the molecular diameters of the gases 201 and 202, the gases 201 and 202 contained in the sample gas can enter the pore 161a. Typically, a gas that has entered the pore 161a may be subjected to a suction force from the wall of the pore 161a present around the gas. That is, the adsorbent 161 having the pore 161a can apply a suction force to the gas that has entered the pore 161a. The adsorbent 161 having the pore 161a can apply a suction force to the gas that has entered the pore 161a, and can thus adsorb the gas more strongly than the adsorbent 161X having no pore 161a. Since the adsorbent 161 having the pore 161a can more strongly suck the gas than the adsorbent 161X having no pore 161a, the desorption temperature of the gas from the adsorbent 161 can be higher than the desorption temperature of the gas from the adsorbent 161X. In other words, because the adsorbent 161 has the pore 161a, the desorption temperature of the gas from the adsorbent 161 can be higher than the desorption temperature of the gas from the adsorbent 161X having no pore 161a. Accordingly, the desorption temperatures of the gases 201 and 202 from the adsorbent 161 having the pore 161a can be higher than the desorption temperatures of the gases 201 and 202 from the adsorbent 161X having no pore 161a.

When the sample gas is a gas generated from feces, as described above, the gas to be detected 201 as illustrated in FIG. 15 can be any one of methane, hydrogen, carbon dioxide, methyl mercaptan, hydrogen sulfide, acetic acid, or trimethylamine. When the sample gas is a gas generated from feces, as described above, the gas not to be detected 202 can be any one of water or ammonia. The molecular diameter of methane, hydrogen, carbon dioxide, methyl mercaptan, hydrogen sulfide, acetic acid, and trimethylamine (which can be the gas to be detected 201) can be larger than the molecular diameter of water and ammonia (which can be the gas not to be detected 202). That is, when the sample gas is a gas generated from feces, the molecular diameter of the gas to be detected 201 can be larger than the molecular diameter of the gas not to be detected 202.

In a configuration as illustrated in FIG. 15 in which the molecular diameter of the gas 201 is larger than the molecular diameter of the gas 202, if the gases 201 and 202 enter the pore 161a, a gap generated between the gas 201 and the wall of the pore 161a can be smaller than a gap generated between the gas 202 and the wall of the pore 161a. Since the gap generated between the gas 201 and the wall of the pore 161a is smaller than the gap generated between the gas 202 and the wall of the pore 161a, the gas to be detected 201 can be more strongly subjected to a suction force from the wall of the pore 161a than the gas not to be detected 202. As described above, because the adsorbent 161 has the pore 161a, the desorption temperature of the gas from the adsorbent 161 can be higher than the desorption temperature of the gas from the adsorbent 161X having no pore 161a. As the suction force to which the gas is subjected from the wall of the pore 161a increases, because the adsorbent 161 has the pore 161a, the degree of increase in the desorption temperature of the gas from the adsorbent 161 can be larger than that in the desorption temperature of the gas from the adsorbent 161X having no pore 161a. Accordingly, when the molecular diameter of the gas 201 is larger than molecular diameter of the gas 202, the degree of increase in the desorption temperature of the gas to be detected 201 is larger than that of the gas not to be detected 202 because of the presence of the pore 161a. With this configuration, the difference between the desorption temperature of the gas to be detected 201 and the desorption temperature of the gas not to be detected 202 in the adsorbent 161 can be larger than the difference between the desorption temperature of the gas to be detected 201 and the desorption temperature of the gas not to be detected 202 in the adsorbent 161X.

For example, in the configuration as illustrated in FIG. 14, the molecular diameter of the gas to be detected is assumed to be larger than the molecular diameter of the gas not to be detected. As described above, the desorption temperature of the gas not to be detected from the adsorbent 161X having no pore 161a is the temperature t101. In contrast, the desorption temperature of the gas not to be detected from the adsorbent 161 having the pore 161a is higher than the temperature t101 and is equal to a temperature till. As described above, furthermore, the desorption temperature of the gas to be detected from the adsorbent 161X having no pore 161a is the temperature t102. In contrast, the desorption temperature of the gas to be detected from the adsorbent 161 having the pore 161a is higher than the temperature t102 and is equal to a temperature t121. As described above, the degree of increase in the desorption temperature of the gas to be detected is larger than that of the gas not to be detected because of the presence of the pore 161a. With this configuration, the difference (t121−t111) between the desorption temperature of the gas to be detected and the desorption temperature of the gas not to be detected in the adsorbent 161 can be larger than the difference (t102−t101) between the desorption temperature of the gas to be detected and the desorption temperature of the gas not to be detected in the adsorbent 161X. In this embodiment, increasing the difference between the desorption temperature of the gas to be detected and the desorption temperature of the gas not to be detected ensures that the gas not to be detected contained in the sample gas can be more reliably removed from the sample gas.

The density of the pore 161a in the adsorbent 161 as illustrated in FIG. 15 may be appropriately selected according to the material of the adsorbent 161 and the type of the gas to be detected. The shape of the pore 161a is an inverted conical shape. However, the shape of the pore 161a is not limited to the inverted conical shape. For example, the shape of the pore 161a may be a cylindrical shape or the like.

The support member 162 as illustrated in FIG. 12 supports the adsorbent 161 near the inlet portion of the concentration tank 160. The support member 162 may be in powder or fiber form containing glass or fluorine resin.

The support member 163 as illustrated in FIG. 12 supports the adsorbent 161 near the outlet portion of the concentration tank 160. The support member 163 may be in powder or fiber form containing glass or fluorine resin.

The heaters 164 as illustrated in FIG. 12 are capable of heating the adsorbent 161. For example, the heaters 164 are energized under the control of the control unit 194 as illustrated in FIG. 13 to heat the adsorbent 161. The heaters 164 are disposed outside the concentration tank 160. The heaters 164 may surround the outer sides of the concentration tank 160. The heaters 164 may be resistance heaters, rubber heaters, or the like.

As illustrated in FIG. 12, the inlet portion of the storage tank 170 is connected to the flow path 134. The outlet portion of the storage tank 170 is connected to the flow path 138. The storage tank 170 is supplied with the purge gas flowing in from the inflow path 121 through the flow paths 130, 133, and 134. The storage tank 170 stores the supplied purge gas. The purge gas stored in the storage tank 170 is supplied to the chamber 180 through the flow paths 138 and 139. The purge gas stored in the storage tank 170 is further supplied to the concentration tank 160 through the flow paths 138, 135, and 132.

The storage tank 70 may be formed by a container or the like having a rectangular parallelepiped shape, a cylindrical shape, a bag shape, or a shape such that it fits in a gap between various components housed inside the housing 110. The storage tank 170 may have a larger capacity than the concentration tank 160. The storage tank 170 includes an adsorbent 171 and support members 172 and 173.

As illustrated in FIG. 12, the adsorbent 171 is placed in the storage tank 170. The adsorbent 171 may contain any material corresponding to the use of the gas detection system 101. The adsorbent 171 may contain, for example, at least any one of activated carbon, silica gel, zeolite, or molecular sieve. The adsorbent 171 may be of a plurality of types or may contain a porous material.

The adsorbent 171 may include an agent that adsorbs a gas to be detected contained in the purge gas. When the air in the toilet room is a purge gas, the purge gas may contain a gas to be detected. Since the adsorbent 171 adsorbs the gas to be detected contained in the purge gas, the purge gas in the storage tank 170 can be purified. When the sample gas is a gas generated from feces, examples of the adsorbent 171 that adsorbs the gas to be detected include activated carbon and molecular sieve. However, the combination of them may be appropriately changed according to the polarity of gas molecules to be adsorbed.

The adsorbent 171 may include an agent that adsorbs a gas not to be detected contained in the purge gas. When the air in the toilet room is a purge gas, the purge gas may contain a gas not to be detected. Since the adsorbent 171 adsorbs the gas not to be detected contained in the purge gas, the purge gas in the storage tank 170 can be purified. When the sample gas is a gas generated from feces, examples of the adsorbent 171 that adsorbs the gas not to be detected include silica gel and zeolite. However, the combination of them may be appropriately changed according to the polarity of gas molecules to be adsorbed.

The support member 172 supports the adsorbent 171 near the inlet portion of the storage tank 170. The support member 172 may be in powder or fiber form containing glass or fluorine resin.

The support member 173 supports the adsorbent 171 near the outlet portion of the storage tank 170. The support member 173 may be in powder or fiber form containing glass or fluorine resin.

As illustrated in FIG. 12, the chamber 180 includes therein a sensor unit 181. The chamber 180 may include a plurality of sensor units 181. The chamber 180 may be divided into a plurality of chambers. The sensor units 181 may be disposed in the resulting plurality of chambers 180. The plurality of chambers 180 may be connected to each other. The chamber 180 is connected to the flow path 137. The chamber 180 is supplied with the sample gas from the flow path 137. The chamber 180 is further connected to the flow path 139. The chamber 180 is supplied with the purge gas from the flow path 139. The chamber 180 is further connected to the discharge path 122. The chamber 180 discharges the sample gas and the purge gas, which have been subjected to detection processing, from the discharge path 122.

As illustrated in FIG. 12, the sensor unit 181 is arranged in the chamber 180. The sensor unit 181 outputs a signal corresponding to the concentration of a specific gas to the control unit 194. The sensor unit 181 may include any sensor such as a semiconductor sensor, a contact combustion sensor, or a solid electrolyte sensor. The sensor unit 181 will be described hereinafter as being configured to output a voltage corresponding to the concentration of the specific gas to the control unit 194 as the signal corresponding to the concentration of the specific gas. However, the signal corresponding to the specific gas, which is output from the sensor unit 181, is not limited to the voltage corresponding to the concentration of the specific gas. For example, the sensor unit 181 may output a current corresponding to the concentration of the specific gas to the control unit 194 as the signal corresponding to the concentration of the specific gas. The specific gas contains a specific gas to be detected and a specific gas not to be detected. When the sample gas is a gas generated from feces, examples of the specific gas to be detected include methane, hydrogen, carbon dioxide, methyl mercaptan, hydrogen sulfide, acetic acid, and trimethylamine. When the sample gas is a gas generated from feces, examples of the specific gas not to be detected include ammonia and water. Each of the plurality of sensor units 181 can output a voltage corresponding to the concentration of at least any one of these gases to the control unit 194.

The circuit board 190 as illustrated in FIG. 13 has mounted therein wiring through which an electrical signal propagates, the storage unit 191, the communication unit 192, the control unit 194, and the like.

The storage unit 191 as illustrated in FIG. 13 can be constituted by, for example, a semiconductor memory, a magnetic memory, or the like. The storage unit 191 stores various kinds of information and a program for operating the gas detection system 101. The storage unit 191 may function as a work memory.

The communication unit 192 as illustrated in FIG. 13 is capable of communicating with the electronic device 103 as illustrated in FIG. 11. The communication unit 192 may be capable of communicating with an external server. The communication method used when the communication unit 192 communicates with the electronic device 103 and the external server may be a short-range wireless communication standard, a wireless communication standard for connecting to a mobile phone network, or a wired communication standard. The short-range wireless communication standard may include, for example, WiFi (registered trademark), Bluetooth (registered trademark), infrared, NFC, and the like. The wireless communication standard for connecting to a mobile phone network may include, for example, LTE, a fourth generation or higher mobile communication system, or the like. Alternatively, the communication method used when the communication unit 192 communicates with the electronic device 103 and the external server may be, for example, a communication standard such as LPWA or LPWAN.

The sensor unit 193 as illustrated in FIG. 13 may include at least any one of an image camera, a personal identification switch, an infrared sensor, a pressure sensor, or the like. The sensor unit 193 outputs a detection result to the control unit 194.

For example, when the sensor unit 193 includes an infrared sensor, the sensor unit 193 detects reflected light from an object irradiated with infrared radiation from the infrared sensor, thereby being able to detect that the subject has entered the toilet room. The sensor unit 193 outputs, as a detection result, a signal indicating that the subject has entered the toilet room to the control unit 194.

For example, when the sensor unit 193 includes a pressure sensor, the sensor unit 193 detects a pressure applied to the toilet seat 102B as illustrated in FIG. 11, thereby being able to detect that the subject has sat on the toilet seat 102B. The sensor unit 193 outputs, as a detection result, a signal indicating that the subject has sat on the toilet seat 102B to the control unit 194.

For example, when the sensor unit 193 includes a pressure sensor, the sensor unit 193 detects a reduction in the pressure applied to the toilet seat 102B as illustrated in FIG. 11, thereby being able to detect that the subject has risen from the toilet seat 102B. The sensor unit 193 outputs, as a detection result, a signal indicating that the subject has risen from the toilet seat 102B to the control unit 194.

For example, when the sensor unit 193 includes an image camera, a personal identification switch, and the like, the sensor unit 193 collects data, such as a face image, the sitting height, and the weight. The sensor unit 193 identifies and detects a person from the collected data. The sensor unit 193 outputs, as a detection result, a signal indicating the identified person to the control unit 194.

For example, when the sensor unit 193 includes a personal identification switch or the like, the sensor unit 193 identifies (detects) a person in response to an operation of the personal identification switch. In this case, personal information may be registered (stored) in the storage unit 191 in advance. The sensor unit 193 outputs, as a detection result, a signal indicating the identified person to the control unit 194.

The control unit 194 as illustrated in FIG. 13 includes one or more processors. The one or more processors may include at least any one of a general-purpose processor that reads a specific program to execute a specific function, or a dedicated processor dedicated to a specific process. The dedicated processor may include an application specific IC (ASIC). The one or more processors may include a programmable logic device (PLD). The PLD may include an FPGA. The control unit 194 may include at least any one of an SoC or an SiP with which the one or more processors cooperate.

<Purge Gas Storage Process>

The control unit 194 can detect that the subject has risen from the toilet seat 102B on the basis of the detection result of the sensor unit 193. The control unit 194 performs control so that the air in the toilet room flows into the inflow path 121 as a purge gas after a first specific time period has elapsed since it was detected that the subject rose from the toilet seat 102B. The control unit 194 performs control so that the purge gas flowing in from the inflow path 121 is stored in the storage tank 170. The first specific time period may be appropriately set in consideration of the time period taken to replace the air in the toilet room with air outside the toilet room by using a ventilation fan or the like in the toilet room after the subject exits the toilet room.

For example, the control unit 194 causes the valve 140 as illustrated in FIG. 12 to connect the inflow path 121 and the flow path 130 to each other, and causes the valve 142 as illustrated in FIG. 12 to connect the flow path 133 and the flow path 134 to each other. Further, the control unit 194 causes the valve 144 as illustrated in FIG. 12 to connect the flow path 138 and the discharge path 124 to each other. In addition, the control unit 194 controls the supply unit 150 to generate a flow of gas from the inflow path 121 toward the discharge path 124 through the flow paths 130, 133, and 134, the storage tank 170, and the flow path 138. As a result of generation of the flow of gas, the air in the toilet room flows into the inflow path 121 as a purge gas. The purge gas flowing in from the inflow path 121 is supplied to the storage tank 170 through the flow paths 130, 133, and 134. Since the purge gas is supplied to the storage tank 170, the residual gas in the storage tank 170 is pushed out to the flow path 138 by the purge gas and discharged from the discharge path 124. The control unit 194 stops the supply unit 150 at a point in time when a second specific time period elapses after the purge gas starts to flow into the inflow path 121. Further, the control unit 194 causes the valve 140 not to connect the inflow path 121 and the flow path 130 to each other, and causes the valve 142 not to connect the flow path 133 and the flow path 134 to each other. In addition, the control unit 194 causes the valve 144 not to connect the flow path 138 and the discharge path 124 to each other. With this configuration, the purge gas from the inflow path 121 is stored in the storage tank 170. The second specific time period may be appropriately set in consideration of the capacity of the storage tank 170 and the like. The purge gas stored in the storage tank 170 can come into contact with the adsorbent 171 in the storage tank 170. Since the purge gas comes into contact with the adsorbent 171, the gas to be detected and the gas not to be detected contained in the purge gas can be adsorbed by the adsorbent 171. Since the gas to be detected and the gas not to be detected contained in the purge gas are adsorbed by the adsorbent 171, the purge gas in the storage tank 170 can be purified.

<Sample Gas Storage and Concentration Process>

The control unit 194 as illustrated in FIG. 13 can detect that the subject has sat on the toilet seat 102B on the basis of the detection result of the sensor unit 193. The control unit 194 performs control so that a gas generated from feces discharged into the toilet bowl 102A flows into the inflow path 120 as a sample gas after a third specific time period has elapsed since it was detected that the subject sat on the toilet seat 102B. The control unit 194 performs control so that the sample gas flowing in from the inflow path 120 passes through the concentration tank 160. For example, the control unit 194 performs control so that the sample gas passes through the concentration tank 160 and is discharged from the discharge path 123. The third specific time period may be appropriately set in consideration of the time period taken until the subject defecates after the subject sits on the toilet seat 102B.

For example, the control unit 194 causes the valve 140 as illustrated in FIG. 12 to connect the inflow path 120 and the flow path 130 to each other, and causes the valve 141 to connect the flow path 131 and the flow path 132 to each other. Further, the control unit 194 causes the valve 143 as illustrated in FIG. 12 to connect the flow path 136 and the discharge path 123 to each other. In addition, the control unit 194 controls the supply unit 150 as illustrated in FIG. 12 to generate a flow of gas from the inflow path 120 toward the discharge path 123 through the flow paths 130, 131, and 132, the concentration tank 160, and the flow path 136. As a result of generation of the flow of gas, the sample gas flowing in from the inflow path 120 passes through the concentration tank 160.

The control unit 194 as illustrated in FIG. 13 performs control so that the sample gas passes through the concentration tank 160 to cause the adsorbent 161 to adsorb the gas to be detected contained in the sample gas. In this case, the control unit 194 maintains the heaters 164 in the non-driven state. Since the heaters 164 are maintained in the non-driven state, the temperature of the adsorbent 161 can be room temperature. The control unit 194 may perform control so that the sample gas passes through the concentration tank 160 for a first specified time period. The first specified time period may be appropriately set in consideration of the amount of the gas to be detected that can be adsorbed by the adsorbent 161. Further, the flow rate of the sample gas passing through the inside of the concentration tank 160 may be appropriately set in consideration of the volumetric capacity of the concentration tank 160, the area of the adsorbent 161, or the like. The control unit 194 may estimate the flow rate of the sample gas from at least any one of a driving voltage, a frequency, or the like of a pump or the like constituting the supply unit 150. The gas detection system 101 may be provided with a flow rate sensor that detects the flow rate of the sample gas. In this configuration, the flow rate sensor outputs a detection signal indicating the flow rate of the sample gas to the control unit 194. The control unit 194 detects the flow rate of the sample gas on the basis of the detection signal output from the flow rate sensor. The control unit 194 may also detect the flow rate of the purge gas in a manner that is the same as or similar to that of the sample gas.

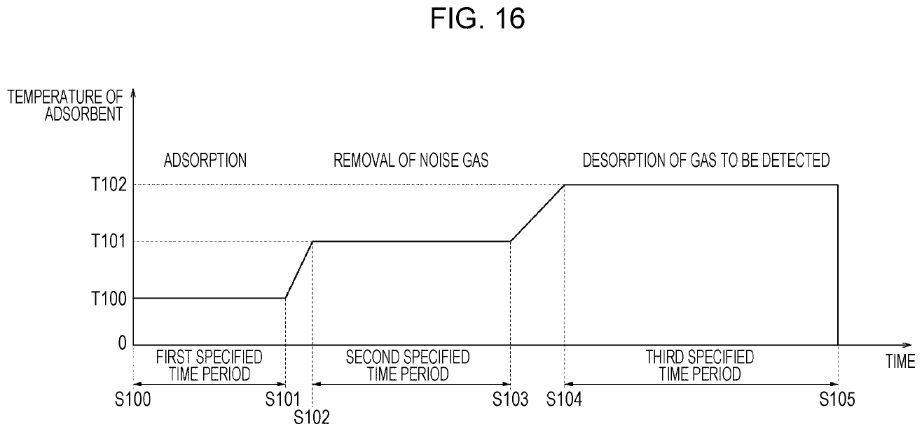

FIG. 16 is a timing chart of an example operation of the gas detection system 101 illustrated in FIG. 11. FIG. 16 illustrates a change in the temperature of the adsorbent 161 with time. The control unit 194 may estimate the temperature of the adsorbent 161 from the current of the heaters 164 or the like. A temperature sensor may be disposed in the vicinity of the adsorbent 161. In this configuration, the temperature sensor outputs a signal indicating the temperature in the vicinity of the adsorbent 161 to the control unit 194. The control unit 194 may acquire the temperature of the adsorbent 161 on the basis of the detection signal output from the temperature sensor.

Time S100 as illustrated in FIG. 16 is a point in time at which the third specific time period elapses after the control unit 194 detects that the subject has sat on the toilet seat 102B. At the time S100, the control unit 194 performs control so that a gas generated from feces discharged into the toilet bowl 102A flows into the inflow path 120 as a sample gas. Further, the control unit 194 performs control so that the sample gas flowing into the inflow path 120 passes through the concentration tank 160. In this case, the control unit 194 maintains the heaters 164 in the non-driven state. Since the heaters 164 are maintained in the non-driven state, the adsorbent 161 is maintained at room temperature T100 after the time S100. The control unit 194 performs control so that the sample gas passes through the concentration tank 160 for the first specified time period from the time S100 to time S101. Since the sample gas passes through the concentration tank 160, the detection target gas contained in the sample gas is adsorbed by the adsorbent 161. The sample gas in which the gas to be detected is adsorbed by the adsorbent 161 is discharged from the discharge path 123. If the sample gas contains a gas not to be detected, the gas not to be detected can also be adsorbed by the adsorbent 161 after the time S100.

The control unit 194 as illustrated in FIG. 13 stops the passage of the sample gas to the concentration tank 160 at a point in time when the first specified time period elapses after the sample gas starts to pass through the concentration tank 160. For example, the control unit 194 stops the supply unit 150 at a point in time when the first specified time period elapses. Further, the control unit 194 causes the valve 141 not to connect the flow path 131 and the flow path 132 to each other, and causes the valve 143 not to connect the flow path 136 and the discharge path 123 to each other. At the point in time when the first specified time period elapses, the control unit 194 brings the heaters 164 into the driven state to increase the temperature of the adsorbent 161.

In FIG. 16, the time S101 is the point in time when the first specified time period elapses after the sample gas starts to pass through the concentration tank 60. At the time S101, the control unit 194 stops the passage of the sample gas to the concentration tank 160. At the time S101, furthermore, the control unit 194 brings the heaters 164 into the driven state. Since the heaters 164 are brought into the driven state at the time S101, the temperature of the adsorbent 161 increases after the time S101.

In response to the temperature of the adsorbent 161 as illustrated in FIG. 12 reaching a temperature T101, the control unit 194 as illustrated in FIG. 13 performs control so that the temperature of the adsorbent 161 is maintained as the temperature T101 for a second specified time period. The second specified time period may be appropriately set in consideration of the amount of the gas not to be detected that can be contained in the sample gas. The temperature T101 may be the desorption temperature of the gas not to be detected that can be contained in the sample gas. As illustrated in FIG. 15, when the adsorbent 161 has the pore 161a, the temperature T101 can be the temperature till as illustrated in FIG. 14. Since the adsorbent 161 is maintained at the temperature T101, the gas not to be detected can be desorbed from the adsorbent 161. The control unit 194 performs control so that the purge gas passes through the concentration tank 160 while performing control so that the temperature of the adsorbent 161 is maintained as the temperature T101. For example, the control unit 194 performs control so that the purge gas that has passed through the concentration tank 160 is discharged from the discharge path 123. With this configuration, the gas not to be detected desorbed from the adsorbent 161 can be discharged from the discharge path 123 together with the purge gas. That is, the gas not to be detected desorbed from the adsorbent 161 can be removed from the concentration tank 160. The flow rate of the purge gas passing through the inside of the concentration tank 160 may be appropriately set in consideration of the volumetric capacity of the concentration tank 160, the area of the adsorbent 161, or the like.

For example, in response to the temperature of the adsorbent 161 as illustrated in FIG. 12 reaching the temperature T101, the control unit 194 causes the valve 140 to connect the inflow path 121 and the flow path 130 to each other, and causes the valve 142 to connect the flow path 133 and the flow path 134 to each other. The control unit 194 further causes the valve 144 to connect the flow path 138 and the flow path 135 to each other, causes the valve 141 to connect the flow path 135 and the flow path 132 to each other, and causes the valve 143 to connect the flow path 136 and the discharge path 123 to each other. In addition, the control unit 194 controls the supply unit 150 to generate a flow of gas from the inflow path 121 toward the discharge path 123 through the flow paths 130, 133, and 134, the storage tank 170, and the flow paths 138, 135, and 132, the concentration tank 160, and the flow path 136. As a result of generation of the flow of the gas, the purge gas passes through the concentration tank 160 and is discharged from the discharge path 123. Since the purge gas passes through the concentration tank 160, the gas not to be detected desorbed from the adsorbent 161 can be removed from the concentration tank 160 and discharged from the discharge path 123 by the purge gas.

In FIG. 16, at time S102, the temperature of the adsorbent 161 reaches the temperature T101. The control unit 194 controls the heaters 164 so that the temperature of the adsorbent 161 is maintained as the temperature T101 for the second specified time period from the time S102 to time S103. Since the temperature of the adsorbent 161 is maintained as the temperature T101 after the time S102, the gas not to be detected can be desorbed from the adsorbent 161. Further, the control unit 194 performs control so that the purge gas passes through the concentration tank 160 and is discharged from the discharge path 123 at the time S102. Since the purge gas passes through the concentration tank 160 and is discharged from the discharge path 123, the desorbed gas not to be detected can be removed from the concentration tank 160 and discharged from the discharge path 123 by the purge gas.

At a point in time when the second specified time period elapses after the temperature of the adsorbent 161 as illustrated in FIG. 12 reaches the temperature T101, the control unit 194 as illustrated in FIG. 13 controls the heaters 164 so that the temperature of the adsorbent 161 increases to the temperature T102. The temperature T102 may be the desorption temperature of the gas to be detected contained in the sample gas. As illustrated in FIG. 15, when the adsorbent 161 has the pore 161a, the temperature T102 can be the temperature t121 as illustrated in FIG. 14. In FIG. 16, the time S103 is a point in time at which the second specified time period elapses. In FIG. 16, at the time S103, the control unit 194 controls the heaters 164 so that the temperature of the adsorbent 161 increases to the temperature T102.

In response to the temperature of the adsorbent 161 reaching the temperature T102, the control unit 194 as illustrated in FIG. 13 controls the heaters 164 so that the temperature of the adsorbent 161 is maintained as the temperature T102. The control unit 194 controls the supply unit 150 so that the purge gas passes through the concentration tank 160 while controlling the heaters 164 so that the temperature of the adsorbent 161 is maintained as the temperature T102. For example, the control unit 194 performs control so that the purge gas passes through the concentration tank 160 and is supplied to the sensor unit 181 in the chamber 180 together with the gas to be detected in the concentration tank 160. With this configuration, the gas to be detected having an increased concentration in the concentration tank 160, that is, the more concentrated sample gas in the concentration tank 160, can be transported to the sensor unit 181 in the chamber 180 by the purge gas. The purge gas is also referred to as a “carrier gas” when used in gas transportation applications. The control unit 194 may perform control so that the purge gas passes through the concentration tank 160 and is supplied to the sensor unit 181 in the chamber 180 together with the gas to be detected in the concentration tank 160 for a third specified time period. The third specified time period may be appropriately set in consideration of the flow rate of the purge gas and the amount of the gas to be detected that can be adsorbed by the adsorbent 161. Further, the flow rate of the purge gas passing through the concentration tank 160 as a carrier gas may be appropriately set in consideration of the amount of the gas to be detected that can be adsorbed by the adsorbent 161, the cross-sectional area of the concentration tank 160, and the like.

For example, in response to the temperature of the adsorbent 161 reaching the temperature T102, the control unit 194 causes the valve 140 as illustrated in FIG. 12 to connect the inflow path 121 and the flow path 130 to each other, and causes the valve 142 to connect the flow path 133 and the flow path 134 to each other. The control unit 194 further causes the valve 144 to connect the flow path 138 and the flow path 135 to each other, causes the valve 141 to connect the flow path 135 and the flow path 132 to each other, and causes the valve 143 to connect the flow path 136 and the flow path 137 to each other. In addition, the control unit 194 controls the supply unit 150 to generate a flow of gas from the inflow path 121 toward the chamber 180 through the flow paths 130, 133, and 134, the storage tank 170, and the flow paths 138, 135, and 132, the concentration tank 160, and the flow paths 136 and 137. As a result of generation of the flow of gas, the purge gas passes through the concentration tank 160 and transports the gas to be detected in the concentration tank 160 to the chamber 180.

In FIG. 16, at time S104, the temperature of the adsorbent 161 reaches the temperature T102. After the time S104, the control unit 194 performs control so that the purge gas passes through the concentration tank 160 and is supplied to the sensor unit 181 in the chamber 180 together with the gas to be detected in the concentration tank 160 while performing control so that the temperature of the adsorbent 161 is maintained as the temperature T102. Further, the control unit 194 performs control so that the purge gas passes through the concentration tank 160 and is supplied to the sensor unit 181 in the chamber 180 together with the gas to be detected in the concentration tank 160 for the third specified time period from the time S104 to time S105.

<Process for Detecting Type and Concentration of Gas>

The control unit 194 performs control so that the purge gas stored in the storage tank 170 is supplied to the sensor unit 181 in the chamber 180. For example, the control unit 194 causes the valve 140 to connect the inflow path 121 and the flow path 130 to each other, causes the valve 142 to connect the flow path 133 and the flow path 134 to each other, and causes the valve 144 to connect the flow path 138 and the flow path 139 to each other. Further, the control unit 194 controls the supply unit 150 to generate a flow of gas from the inflow path 121 toward the chamber 180 through the flow paths 130, 133, and 134, the storage tank 170, and the flow paths 138 and 139. As a result of generation of the flow of gas, the purge gas stored in the storage tank 170 is supplied to the sensor unit 181 in the chamber 180.

The control unit 194 performs control so that the purge gas passes through the concentration tank 160 and is supplied to the sensor unit 181 in the chamber 180 together with the gas to be detected in the concentration tank 160 in the way described in the <Sample Gas Storage and Concentration Process> section described above.

The control unit 194 performs control so that the purge gas stored in the storage tank 170 and the sample gas concentrated in the concentration tank 160 are alternately supplied to the sensor unit 181 in the chamber 180. The control unit 194 alternately supplies the purge gas and the concentrated sample gas to the chamber 180 to acquire a voltage waveform from the sensor unit 181 in the chamber 180. The control unit 194 detects the type and concentration of a gas contained in the sample gas by, for example, machine learning for the acquired voltage waveform. The control unit 194 may transmit the detected type and concentration of the gas to the electronic device 103 via the communication unit 192 as a detection result.

[Operation of Gas Detection System]

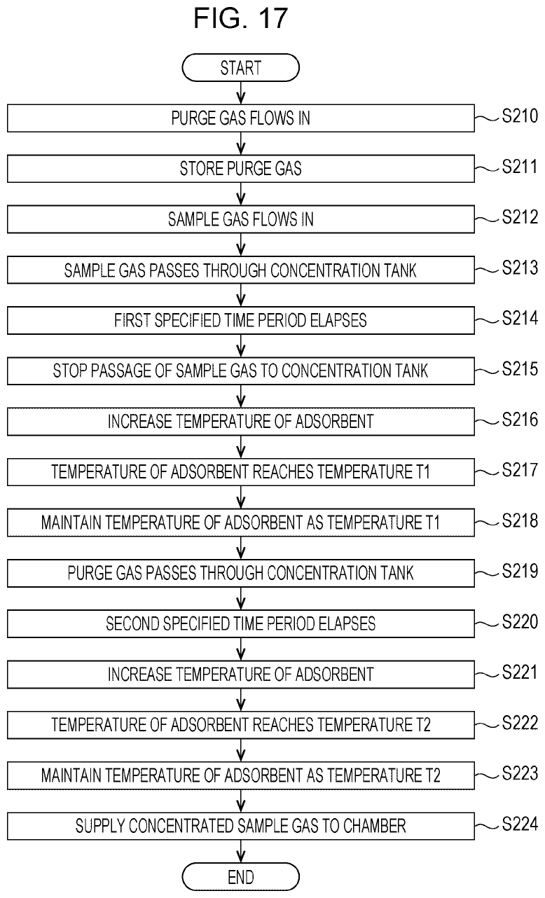

FIG. 17 is a flowchart of an example operation of the gas detection system 101 illustrated in FIG. 11 during gas concentration. The control unit 194 may start a process as illustrated in FIG. 17 after the first specific time period elapses after it is detected that the subject has risen from the toilet seat 102B on the basis of the detection result of the sensor unit 193.

The control unit 194 performs control so that the air in the toilet room flows into the inflow path 121 as a purge gas (step S210). The control unit 194 performs control so that the purge gas flowing into the inflow path 121 is stored in the storage tank 170 (step S211).

The control unit 194 performs control so that a gas generated from feces discharged into the toilet bowl 102A flows into the inflow path 120 as a sample gas after the third specific time period has elapsed since it was detected that the subject sat on the toilet seat 102B (step S212). The control unit 194 performs control so that the sample gas flowing in from the inflow path 120 passes through the concentration tank 160 for the first specified time period (step S213). In the processing of step S213, the control unit 194 maintains the heaters 164 in the non-driven state.

The control unit 194 detects the lapse of the first specified time period after the sample gas starts to pass through the concentration tank 160 (step S214). The control unit 194 stops the passage of the sample gas to the concentration tank 160 at the point in time at which the first specified time period elapses (step S215). The control unit 194 controls the heaters 164 so that the temperature of the adsorbent 161 increases (step S216).

The control unit 194 detects the temperature of the adsorbent 161 reaching the temperature T101 (step S217). In response to the temperature of the adsorbent 161 reaching the temperature T101, the control unit 194 controls the heaters 164 so that the temperature of the adsorbent 161 is maintained as the temperature T101 for the second specified time period (step S218). The control unit 194 performs control so that the purge gas passes through the concentration tank 160 while performing control so that the temperature of the adsorbent 161 is maintained as the temperature T101 (step S219). In the processing of step S219, the control unit 194 performs control so that the purge gas that has passed through the concentration tank 160 is discharged from the discharge path 123.

The control unit 194 detects the lapse of the second specified time period after the temperature of the adsorbent 161 reaches the temperature T101 (step S220). The control unit 194 controls the heaters 164 so that the temperature of the adsorbent 161 increases to the temperature T102 at the point in time at which the second specified time period elapses (step S221).

The control unit 194 detects the temperature of the adsorbent 161 reaching the temperature T102 (step S222). The control unit 194 performs control so that the purge gas passes through the concentration tank 160 (step S224) while controlling the heaters 64 so that the temperature of the adsorbent 161 is maintained as the temperature T102 (step S223). In the processing of step S224, the control unit 194 performs control so that the purge gas passes through the concentration tank 160 and is supplied to the sensor unit 181 in the chamber 180 together with the gas to be detected in the concentration tank 160. After the processing of step S224 ends, the control unit 194 ends the gas concentration process.

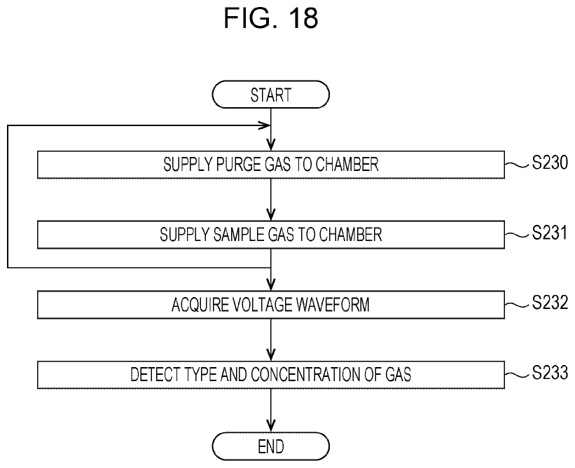

FIG. 18 is a flowchart of an example operation of the gas detection system 101 illustrated in FIG. 11 during detection of the type and concentration of a gas.

The control unit 194 performs control so that the purge gas stored in the storage tank 170 is supplied to the sensor unit 181 in the chamber 180 (step S230). The control unit 194 executes the process as illustrated in FIG. 17 to perform control so that the sample gas concentrated in the concentration tank 160 is supplied to the sensor unit 181 in the chamber 180 (step S231).

The control unit 194 alternately executes the processing of step S230 and the processing of step S231 to perform control so that the purge gas in the storage tank 170 and the sample gas in the concentration tank 160 are alternately supplied to the sensor unit 181 in the chamber 180.

The control unit 194 alternately supplies the purge gas and the concentrated sample gas to the chamber 180 to acquire a voltage waveform from the sensor unit 181 in the chamber 180 (step S232). The control unit 194 detects the type and concentration of a gas contained in the sample gas by, for example, machine learning for the acquired voltage waveform (step S233). After the processing of step S233 ends, the control unit 194 ends the process for detecting the type and concentration of the gas.

As described above, in the gas detection system 101 according to the third embodiment, as illustrated in FIG. 15, the pore size of the pore 161a of the adsorbent 161 is larger than the molecular diameter of the gas to be detected 201 and the molecular diameter of the gas not to be detected 202. Further, the molecular diameter of the gas to be detected 201 is larger than the molecular diameter of the gas not to be detected 202. With this configuration, as described above, the difference (t121−t111) between the desorption temperature of the gas to be detected and the desorption temperature of the gas not to be detected in the adsorbent 161 can be increased. That is, the difference between the temperature T101 and the temperature T102 as illustrated in FIG. 16 can be increased. Since the difference between the temperature T101 and the temperature T102 is increased, the gas not to be detected can be more reliably removed from the sample gas. Since the gas not to be detected is more reliably removed from the sample gas, the gas detection system 101 can more accurately detect the type and concentration of the gas to be detected contained in the sample gas. Accordingly, the third embodiment can provide the improved gas detection system 101.

### Fourth Embodiment

A gas detection system according to a fourth embodiment of the present disclosure will be described hereinafter. The gas detection system according to the fourth embodiment can adopt a configuration that is the same as or similar to that of the gas detection system 101 according to the third embodiment. The following mainly describes differences from the third embodiment with reference to FIGS. 11 to 13.

When the sample gas is to be concentrated in the concentration tank 160 as illustrated in FIG. 12, the control unit 194 as illustrated in FIG. 13 controls the supply unit 150 so that the sample gas passes through the concentration tank 160 as illustrated in FIG. 12 in a manner that is the same as or similar to that in the third embodiment. In this case, in the fourth embodiment, the control unit 194 performs control so that the temperature of the adsorbent 161 is maintained as the temperature T101. As described above, the temperature T101 may be the desorption temperature of the gas not to be detected that can be contained in the sample gas. Since the temperature of the adsorbent 161 is maintained as the temperature T101 when the sample gas passes through the concentration tank 160 as illustrated in FIG. 12, the gas not to be detected contained in the sample gas can be discharged from the discharge path 123 as illustrated in FIG. 12 without being adsorbed by the adsorbent 161 as illustrated in FIG. 12. Since the gas not to be detected contained in the sample gas is discharged from the discharge path 123 as illustrated in FIG. 12 without being adsorbed by the adsorbent 161 as illustrated in FIG. 12, in the fourth embodiment, for example, the second specified time period for removing the noise gas as illustrated in FIG. 16 can be reduced. With this configuration, in the fourth embodiment, the time taken to concentrate the sample gas in the concentration tank 160 can be shortened.

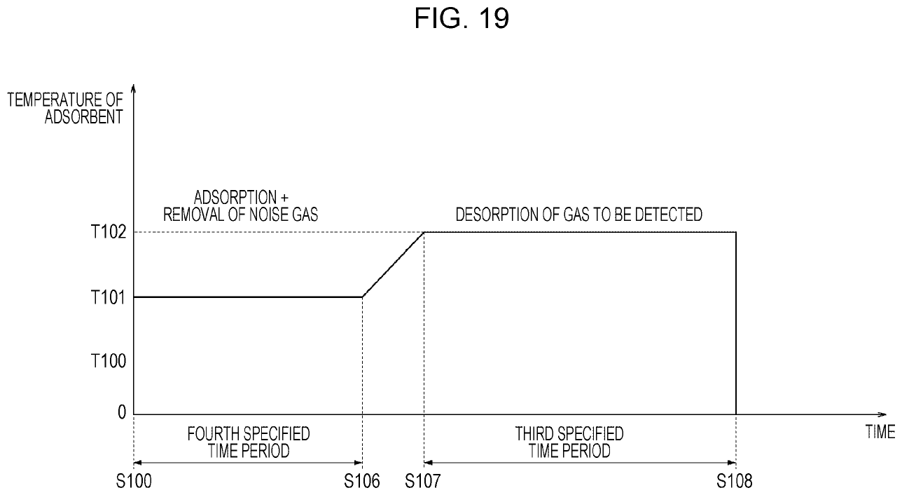

FIG. 19 is timing chart of an example operation of the gas detection system 101 according to the fourth embodiment of the present disclosure. FIG. 19 illustrates a change in the temperature of the adsorbent 161 with time.

Time S100 as illustrated in FIG. 19 is a point in time at which the third specific time period elapses after the control unit 194 detects that the subject has sat on the toilet seat 102B. At the time S100, the control unit 194 performs control so that a gas generated from feces discharged into the toilet bowl 102A flows into the inflow path 120 as a sample gas. Further, the control unit 194 performs control so that the sample gas flowing into the inflow path 120 passes through the concentration tank 160. Further, the control unit 194 controls the heaters 164 so that the temperature of the adsorbent 161 is maintained as the temperature T101 after the time S100. The control unit 194 may control the heaters 164 before the time S100 to increase the temperature of the adsorbent 161 in advance. For example, the control unit 194 may switch the heaters 164 from the non-driven state to the driven state at the point in time when the control unit 194 detects that the subject has sat on the toilet seat 102B. Since the temperature of the adsorbent 161 is maintained as the temperature T101 when the sample gas passes through the concentration tank 160, the gas not to be detected contained in the sample gas can be discharged from the discharge path 123 as illustrated in FIG. 12 without being adsorbed by the adsorbent 161. Further, since the temperature of the adsorbent 161 is maintained as the temperature T101 lower than the temperature T102, the gas to be detected contained in the sample gas can be adsorbed by the adsorbent 161.

The control unit 194 as illustrated in FIG. 13 may control the heaters 164 so that the temperature of the adsorbent 161 is maintained as the temperature T101 while controlling the supply unit 150 so that the sample gas passes through the concentration tank 160 for a fourth specified time period. In FIG. 19, the fourth specified time period is a time period from the time S100 to time S106. The fourth specified time period may be appropriately set in consideration of at least any one of the amount of the gas to be detected that can be adsorbed by the adsorbent 161 or the amount of the gas not to be detected that can be contained in the sample gas. The fourth specified time period may be a time period equivalent to the first specified time period or the second specified time period as illustrated in FIG. 16. Alternatively, the fourth specified time period may be set independently of the first specified time period and the second specified time period.

At a point in time when the fourth specified time period elapses after the sample gas starts to pass through the concentration tank 160 as illustrated in FIG. 12, the control unit 194 as illustrated in FIG. 13 controls the heaters 164 so that the temperature of the adsorbent 161 increases. The control unit 194 controls the heaters 164 so that the temperature of the adsorbent 161 increases to the temperature T102 in a manner that is the same as or similar to that in the third embodiment. In response to the temperature of the adsorbent 161 reaching the temperature T102, the control unit 194 controls the heaters 164 so that the temperature of the adsorbent 161 is maintained as the temperature T102 in a manner that is the same as or similar to that in the third embodiment. The control unit 194 performs control so that the purge gas passes through the concentration tank 160 and is supplied to the sensor unit 181 in the chamber 180 together with the gas to be detected in the concentration tank 160 while controlling the heaters 164 so that the temperature of the adsorbent 161 is maintained as the temperature T102 in a manner that is the same as or similar to that in the third embodiment.

In FIG. 19, the time S106 is a point in time at which the fourth specified time period elapses. At the time S106, the control unit 194 controls the heaters 164 so that the temperature of the adsorbent 161 increases to the temperature T102. At time S107, the temperature of the adsorbent 161 reaches the temperature T102. At the time S107, the control unit 194 controls the supply unit 150 so that the purge gas passes through the concentration tank 160 while controlling the heaters 164 so that the temperature of the adsorbent 161 is maintained as the temperature T102. The control unit 194 performs control so that the purge gas passes through the concentration tank 160 and is supplied to the sensor unit 181 in the chamber 180 together with the gas to be detected in the concentration tank 160.

The control unit 194 as illustrated in FIG. 13 may perform control so that the purge gas passes through the concentration tank 160 and is supplied to the sensor unit 181 in the chamber 180 together with the gas to be detected in the concentration tank 160 for the third specified time period in a manner that is the same as or similar to that in the third embodiment. In FIG. 19, the third specified time period is a time period from the time S107 to time S108.

[Operation of Gas Detection System]

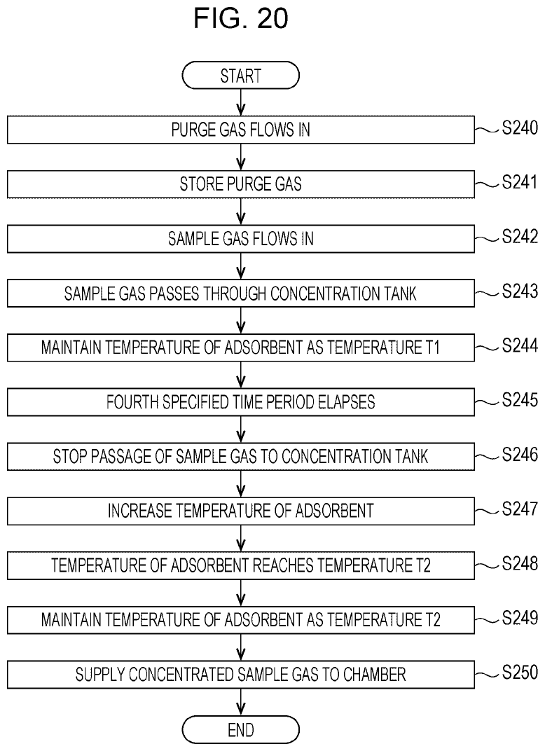

FIG. 20 is a flowchart of an example operation of the gas detection system 101 according to the fourth embodiment of the present disclosure during gas concentration. The control unit 194 may start a process as illustrated in FIG. 20 after the first specific time period elapses after it is detected that the subject has risen from the toilet seat 102B on the basis of the detection result of the sensor unit 193.

The control unit 194 executes the processing of steps S240, S241, S242, and S243 in a way that is the same as or similar to that of the processing of steps S210, S211, S212, and S213 as illustrated in FIG. 17. When executing the processing of step S243, the control unit 194 controls the heaters 164 so that the temperature of the adsorbent 161 is maintained as the temperature T101 (step S244).

The control unit 194 detects the lapse of the fourth specified time period after the processing of step S213 is executed (step S245). The control unit 194 executes the processing of step S246 in a way that is the same as or similar to that of the processing of step S215 as illustrated in FIG. 17.

The control unit 194 executes the processing of steps S247, S248, S249, and S250 in a way that is the same as or similar to that of the processing of steps S221, S222, S223, and S224 as illustrated in FIG. 17. After the processing of step S250 ends, the control unit 194 ends the gas concentration process.

As described above, in the gas detection system 101 according to the fourth embodiment, when controlling the supply unit 150 so that the sample gas passes through the concentration tank 160 as illustrated in FIG. 12, the control unit 194 controls the heaters 164 so that the temperature of the adsorbent 161 is maintained as the temperature T101. With this configuration, in the gas detection system 101 according to the fourth embodiment, the time taken to concentrate the sample gas in the concentration tank 160 can be shortened. In the fourth embodiment, since the time taken to concentrate the sample gas in the concentration tank 160 is shortened, the detection time period in the gas detection system 101 can be shortened.

Other advantages and configurations of the gas detection system 101 according to the fourth embodiment are the same as or similar or to those of the gas detection system 101 according to the third embodiment.

### Fifth Embodiment

A fifth embodiment describes the pore size of the pore 161a of the adsorbent 161. The adsorbent 161 according to the fifth embodiment can be applied to the third embodiment and the fourth embodiment.

As described above, the pore size of the pore 161a of the adsorbent 161 as illustrated in FIG. 15 is larger than the molecular diameter of the gas to be detected 201. Further, the pore size of the pore 161a of the adsorbent 161 may be less than or equal to twice the molecular diameter of the gas to be detected 201.

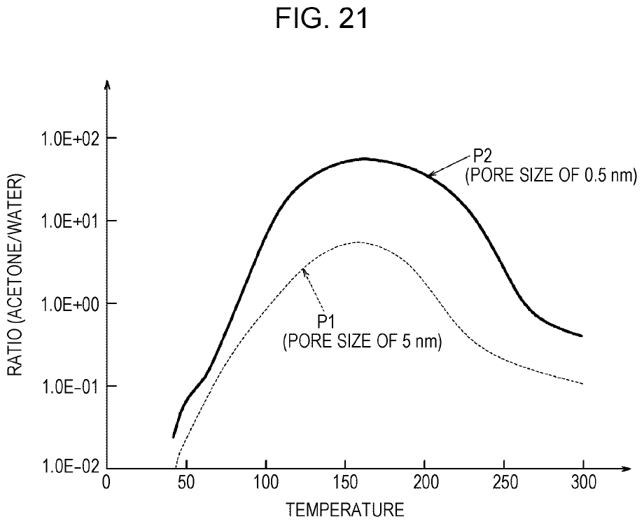

FIG. 21 is a schematic graph illustrating the relationship between the temperature of an adsorbent and the concentration of a gas desorbed from the adsorbent in the fifth embodiment of the present disclosure. Specifically, a curve P1 and a curve P2 are obtained by plotting the ratio of the gas to be detected to the gas not to be detected, desorbed from the adsorbent 161, while changing the temperature of the adsorbent 161 that has adsorbed the gas to be detected and the gas not to be detected. In FIG. 21, acetone was used as a gas to be detected, and water was used as a gas not to be detected. The molecular diameter of acetone is 0.467 nm. The molecular diameter of water is 0.265 nm. In the curve P1, the adsorbent 161 with the pore 161a having a pore size of 5 nm was used. In the curve P2, the adsorbent 161 with the pore 161a having a pore size of 0.5 nm was used. In both the curve P1 and the curve P2, the pore size of the pore 161a of the adsorbent 161 used is larger than the molecular diameter of acetone, namely, 0.467 nm, and the molecular diameter of water, namely, 0.265 nm.

A peak value indicated by the curve P2 was about 10 times larger than a peak value indicated by the curve P1. These results indicate that a sample gas in which the detection target has a higher concentration is obtained when the adsorbent 161 with the pore 161a having a pore size of 0.5 nm is used than when the adsorbent 161 with the pore 161a having a pore size of 5 nm is used. The pore size of the pore 161a of the adsorbent 161 used in the curve P1, namely, 5 nm, is larger than twice the molecular diameter of acetone, namely, 0.467 nm (pore size of 5 nm>molecular diameter of 0.467 nm×2). The pore size of the pore 161a of the adsorbent 161 used in the curve P2, namely, 0.5 nm, is less than or equal to twice the molecular diameter of acetone, namely, 0.467 nm (pore size of 0.5 nm molecular diameter of 0.467 nm×2). In a case where the pore size of the pore 161a is less than or equal to twice the molecular diameter of acetone, the gap generated between acetone and the pore 161a can be narrower than in a case where the pore size of the pore 161a is larger than twice the molecular diameter of acetone. Accordingly, in a case where the pore size of the pore 161a is less than or equal to twice the molecular diameter of acetone, acetone may be less likely to exit the pore 161a at temperatures, except for the desorption temperature of acetone, than in a case where the pore size of the pore 161a is larger than twice the molecular diameter of acetone. In contrast, both the pore size of the pore 161a of the adsorbent 161 used in the curve P1, namely, 5 nm, and the pore size of the pore 161a of the adsorbent 161 used in the curve P2, namely, 0.5 nm, are larger than about 1.8 times the molecular diameter of water, namely, 0.265 nm. Thus, water can mostly be desorbed from the adsorbent 161 at the desorption temperature of water in both the case where the pore size of the pore 161a is 5 nm and the case where the pore size of the pore 161a is 0.5 nm. With this configuration, the peak value indicated by the curve P2 is considered to be about ten times larger than the peak value indicated by the curve P1. Accordingly, in a case where the pore size of the pore 161a is larger than the molecular diameter of the gas to be detected and is less than or equal to twice the molecular diameter of the gas to be detected (molecular diameter of 0.467 nm<pore size of 0.5 nm molecular diameter of 0.467 nm×2), a sample gas in which the detection target has a high concentration is obtained.

As indicated by the curve P1, in a case where the pore size of the pore 161a of the adsorbent 161 was 5 nm, the desorption temperature of acetone was about 162 degrees. As indicated by the curve P2, in a case where the pore size of the pore 161a of the adsorbent 161 was 0.5 nm, the desorption temperature of acetone was about 167 degrees. In both the case where the pore size of the pore 161a was 5 nm and the case where the pore size of the pore 161a was 0.5 nm, the desorption temperature of acetone was higher than that in a case where the adsorbent had no pore 161a. Further, the desorption temperature of acetone (167 degrees) in a case where the pore size of the pore 161a is 0.5 nm is higher by about 5 degrees than the desorption temperature of acetone (162 degrees) in a case where the pore size of the pore 161a is 5 nm. These results indicate that the desorption temperature of the gas to be detected is high in a case where the pore size of the pore 161a is larger than the molecular diameter of the gas to be detected and is less than or equal to twice the molecular diameter of the gas to be detected (molecular diameter of 0.467 nm<pore size of 0.5 nm molecular diameter of 0.467 nm×2).

(Modifications of Third Embodiment to Fifth Embodiment)

Modifications of the third embodiment to the fifth embodiment will be described hereinafter.

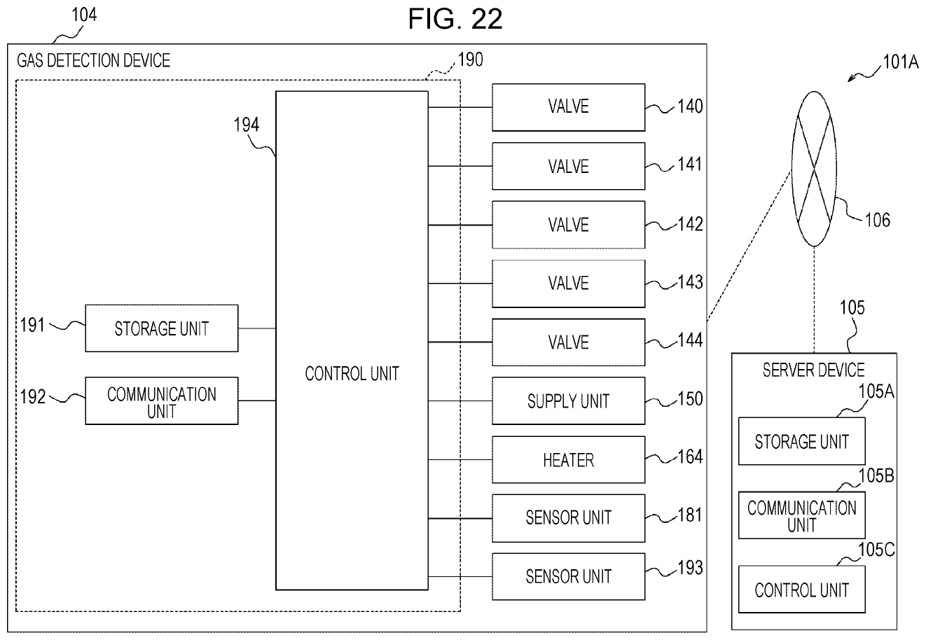

For example, in the third embodiment to the fifth embodiment described above, as illustrated in FIG. 13, the gas detection system 101 has been described as a single device. However, the gas detection system according to the present disclosure is not limited to the single device. The gas detection system according to the present disclosure may include a plurality of independent devices. For example, the third embodiment to the fifth embodiment described above may adopt a gas detection system 101A having a configuration as illustrated in FIG. 22.

As illustrated in FIG. 22, the gas detection system 101A includes a gas detection device 104 and a server device 105. The gas detection device 104 and the server device 105 are capable of communicating with each other via a network 106. A portion of the network 106 may be wired or wireless. The gas detection device 104 has a configuration that is the same as or similar to the configuration of the gas detection system 101 as illustrated in FIG. 12 and FIG. 13. The server device 105 includes a storage unit 105A, a communication unit 105B, and a control unit 105C. The control unit 105C is capable of executing the processes of the control unit 194 as illustrated in FIG. 13 described above. For example, the control unit 105C is capable of controlling the supply unit 150 so that the sample gas concentrated in the concentration tank 160 is supplied to the sensor unit 181 in the chamber 180.

The configurations of the third embodiment to the fifth embodiment described above can be summarized in the following appendices.

(Appendix 1)

A gas detection system including:

a sensor unit that outputs a signal corresponding to a concentration of a specific gas;

a concentration unit having therein an adsorbent having a pore; and

a supply unit capable of supplying a sample gas to the concentration unit, wherein

the pore has a larger pore size than an effective molecular diameter of a gas to be detected and an effective molecular diameter of a gas not to be detected, the gas to be detected and the gas not to be detected being contained in the sample gas, and

the effective molecular diameter of the gas to be detected is larger than the effective molecular diameter of the gas not to be detected.

(Appendix 2)

The gas detection system according to appendix 1, wherein

the pore size is less than or equal to twice the effective molecular diameter of the gas to be detected.

(Appendix 3)

The gas detection system according to appendix 1 or 2, wherein

the gas to be detected includes at least one of methane, hydrogen, carbon dioxide, methyl mercaptan, hydrogen sulfide, acetic acid, or trimethylamine, and

the gas not to be detected includes at least one of water or ammonia.

(Appendix 4)

The gas detection system according to any one of appendices 1 to 3, further including

a heater capable of heating the adsorbent.

(Appendix 5)

The gas detection system according to appendix 4, further including

a control unit capable of controlling the supply unit so that the sample gas, which is concentrated in the concentration unit, is supplied to the sensor unit, wherein

when the sample gas is to be concentrated in the concentration unit, the control unit controls the supply unit so that the sample gas passes through the concentration unit, and then controls the heater so that a temperature of the adsorbent increases to a desorption temperature of the gas to be detected.

(Appendix 6)

The gas detection system according to appendix 5, wherein

when controlling the supply unit so that the sample gas passes through the concentration unit, the control unit maintains the heater in a non-driven state.

(Appendix 7)

The gas detection system according to appendix 5, wherein

when controlling the supply unit so that the sample gas passes through the concentration unit, the control unit controls the heater so that the temperature of the adsorbent is maintained as a desorption temperature of the gas not to be detected.

The drawings describing embodiments according to the present disclosure are schematic ones. Dimensional ratios and the like in the drawings do not necessarily match the actual ones.

While embodiments according to the present disclosure have been described with reference to the drawings and examples, it should be noted that various modifications or changes can be easily made by a person skilled in the art on the basis of the present disclosure. Accordingly, it should be noted that these modifications or changes fall within the scope of the present disclosure. For example, the functions and the like included in each component or the like can be rearranged in any manner that is not logically contradictory, and a plurality of components may be combined into one or divided.

In the present disclosure, descriptions such as “first” and “second” are identifiers for distinguishing the respective configurations. The configurations distinguished by the descriptions such as “first” and “second” in the present disclosure may be interchangeably numbered. The identifiers are exchanged simultaneously. Even after the identifiers are exchanged, the respective configurations are distinguishable. The identifiers may be deleted. Configurations without identifiers are distinguished using reference numerals. Only the description of identifiers such as “first” and “second” in the present disclosure should not be used for interpreting the order of the configurations or as a basis of the presence of identifiers with smaller numbers.

### REFERENCE SIGNS LIST

1, 1A gas detection system
2 toilet
2A toilet bowl
2B toilet seat
3 electronic device
3A display unit
4 gas detection device
5 server device
5A storage unit
5B communication unit
5C control unit
6 network
10 housing
20, 21 inflow path
22, 23, 24 discharge path
30, 31, 32, 33, 34, 35, 36, 37, 38, 39 flow path
40, 41, 42, 43, 44 valve
50 supply unit
60 concentration tank (concentration unit)
61 adsorbent
62, 63 support member
64 heater
70 storage tank (reservoir)
71 adsorbent
72, 73 support member
80 chamber
81 sensor unit
90 circuit board
91 storage unit
92 communication unit
93 sensor unit
94 control unit
101, 101A gas detection system
102 toilet
102A toilet bowl
102B toilet seat
103 electronic device
103A display unit
104 gas detection device
105 server device
105A storage unit
105B communication unit
105C control unit
106 network
110 housing
120, 121 inflow path
122, 123, 124 discharge path
130, 131, 132, 133, 134, 135, 136, 137, 138, 139 flow
path
140, 141, 142, 143, 144 valve
150 supply unit
160 concentration tank (concentration unit)
161 adsorbent
161a pore
162, 163 support member
164 heater
170 storage tank (reservoir)
171 adsorbent
172, 73 support member
180 chamber
181 sensor unit
190 circuit board
191 storage unit
192 communication unit
193 sensor unit
194 control unit
201, 202 gas

1. A gas detection system comprising:
a sensor unit that outputs a signal corresponding to a concentration of a specific gas;
a concentration unit having an adsorbent that adsorbs a gas to be detected;
a supply unit capable of supplying a sample gas and a purge gas to the concentration unit;
a heater capable of heating the adsorbent; and
a control unit that controls the supply unit so that the sample gas passes through the concentration unit and then controls the supply unit so that the purge gas passes through the concentration unit while controlling the heater so that a temperature of the adsorbent increases, wherein
the control unit
stops passage of the purge gas to the concentration unit from a first point in time to a second point in time later than the first point in time, the first point in time being a point in time before or at which the temperature of the adsorbent reaches a desorption temperature of the gas to be detected, and
the control unit controls the supply unit after the second point in time so that the purge gas passes through the concentration unit and is supplied to the sensor unit together with the gas to be detected in the concentration unit.

2. The gas detection system according to claim 1, wherein
the control unit controls the supply unit so that the sample gas passes through the concentration unit for a first time period.

3. The gas detection system according to claim 2, wherein
when controlling the heater so that the temperature of the adsorbent increases, in response to the temperature of the adsorbent reaching a desorption temperature of a gas not to be detected, the control unit controls the supply unit so that the purge gas passes through the concentration unit while controlling the heater so that the temperature of the adsorbent is maintained as the desorption temperature for a second time period.

4. The gas detection system according to claim 1, wherein
the first point in time is a point in time at which the temperature of the adsorbent reaches a temperature set based on a temperature at which the gas to be detected starts to be desorbed from the adsorbent, and
the second point in time is a point in time at which a third time period elapses after the temperature of the adsorbent reaches the desorption temperature of the gas to be detected.

5. The gas detection system according to claim 1, wherein
the first point in time is a point in time at which the temperature of the adsorbent reaches a temperature set based on a temperature at which the gas to be detected starts to be desorbed from the adsorbent, and
the second point in time is a point in time at which the temperature of the adsorbent reaches the desorption temperature of the gas to be detected.

6. The gas detection system according to claim 1, wherein
the first point in time is a point in time at which the temperature of the adsorbent reaches the desorption temperature of the gas to be detected, and
the second point in time is a point in time at which a third time period elapses after the temperature of the adsorbent reaches the desorption temperature of the gas to be detected.

7. The gas detection system according to claim 1, wherein
the first point in time is a point in time at which the temperature of the adsorbent exceeds a desorption temperature of a gas not to be detected, and
the second point in time is a point in time at which a third time period elapses after the temperature of the adsorbent reaches the desorption temperature of the gas to be detected.

8. The gas detection system according to claim 1, wherein
when controlling the supply unit so that the sample gas passes through the concentration unit, the control unit controls the supply unit so that a flow rate of the sample gas passing through the concentration unit is a first flow rate, and
when controlling the supply unit so that the purge gas passes through the concentration unit from the second point in time, the control unit controls the supply unit so that a flow rate of the purge gas passing through the concentration unit is a second flow rate smaller than the first flow rate and exhausts the purge gas that has passed through the concentration unit without supplying the purge gas to the sensor unit until a fourth time period elapses after the purge gas starts to pass through the concentration unit, and after the fourth time period elapses, the control unit controls the supply unit so that the purge gas passes through the concentration unit and is supplied to the sensor unit together with the gas to be detected in the concentration unit.

9. The gas detection system according to claim 1, wherein
when the control unit controls the supply unit so that the sample gas passes through the concentration unit, a flow rate of the sample gas passing through the concentration unit is a first flow rate, and
when the control unit controls the supply unit so that the purge gas passes through the concentration unit from the second point in time, a flow rate of the purge gas passing through an inside of the concentration unit is a third flow rate larger than the first flow rate.

10. The gas detection system according to claim 1, wherein
the adsorbent has a pore,
the pore has a larger pore size than an effective molecular diameter of a gas to be detected and an effective molecular diameter of a gas not to be detected, the gas to be detected and the gas not to be detected being contained in the sample gas, and
the effective molecular diameter of the gas to be detected is larger than the effective molecular diameter of the gas not to be detected.

11. The gas detection system according to claim 10, wherein
the pore size is less than or equal to twice the effective molecular diameter of the gas to be detected.

12. The gas detection system according to claim 10, wherein
the gas to be detected includes at least one of methane, hydrogen, carbon dioxide, methyl mercaptan, hydrogen sulfide, acetic acid, or trimethylamine, and
the gas not to be detected includes at least one of water or ammonia.

13. The detection system according to claim 10, wherein
when the sample gas is to be concentrated in the concentration unit, the control unit controls the supply unit so that the sample gas passes through the concentration unit, and then controls the heater so that the temperature of the adsorbent increases to a desorption temperature of the gas to be detected.

14. The gas detection system according to claim 13, wherein
when controlling the supply unit so that the sample gas passes through the concentration unit, the control unit maintains the heater in a non-driven state.

15. The gas detection system according to claim 13, wherein
when controlling the supply unit so that the sample gas passes through the concentration unit, the control unit controls the heater so that the temperature of the adsorbent is maintained as a desorption temperature of the gas not to be detected.
